(19) United States
(12) Patent Application Publication
(10) Pub. No.: US 2022/0211752 A1
(43) Pub. Date: Jul. 7, 2022

(54) METHIONINE AND CYSTEINE DEPRIVATION DIET AND FORMULATIONS TO INCREASE EFFECTIVENESS OF CANCER THERAPY

(71) Applicant: THE TRUSTEES OF COLUMBIA UNIVERSITY IN THE CITY OF NEW YORK, New York, NY (US)

(72) Inventors: Pavan Upadhyayula, New York, NY (US); Dominique Higgins, New York, NY (US); Jeffrey N. Bruce, New York, NY (US); Simon Cheng, New York, NY (US); Kunal Chaudhary, New York, NY (US); Peter Canoll, New York, NY (US); Brent Stockwell, New York, NY (US); Athanassios Dovas, New York, NY (US)

(21) Appl. No.: 17/535,969

(22) Filed: Nov. 26, 2021

Related U.S. Application Data
(60) Provisional application No. 63/118,339, filed on Nov. 25, 2020.
(60) Provisional application No. 63/227,219, filed on Jul. 29, 2021.

(51) Int. Cl. A61K 33/26; A61K 31/355; A61K 31/198; A61K 33/04; A61K 38/05; A61K 31/201; A61K 31/202; A61P 35/00

(52) U.S. Cl. CPC A61K 33/26; A61K 31/355; A61K 31/198; A61P 35/00; A61K 38/05; A61K 31/201; A61K 31/202; A61K 33/04

(57) ABSTRACT

Ferroptosis (cell death mediated by iron-dependent lipid peroxide accumulation) results from lipid peroxidation of phospholipids containing polyunsaturated fatty acyl moieties. Glutathione, the key cellular antioxidant capable of inhibiting lipid peroxidation via the activity of the enzyme glutathione peroxidase 4 (GPX-4), is generated directly from the sulfur-containing aminoacid cysteine, and indirectly from methionine via the transsulfuration pathway. Cysteine and methionine deprivation (CMD) in the diet can synergistically increase RSL3-mediated cell death and lipid peroxidation in both murine and human glioma cell lines and in ex-vivo organotypic slice cultures. A cysteine-depleted, methionine-restricted diet can improve survival in an syngeneic orthotopic murine glioma model. This CMD diet leads to profound in-vivo metabolomic, proteomic and lipidomic alterations, leading to improvements in the efficacy of ferroptotic therapies in glioma treatment with a non-invasive dietary modification.

### CROSS-REFERENCE TO RELATED APPLICATIONS

This application claims the benefit of U.S. provisional application Ser. No. 63/118,339, filed 25 Nov. 2020 and U.S. provisional application Ser. No. 63/227,219, filed 29 Jul. 2021. The entire contents of these applications are hereby incorporated by reference as if fully set forth herein.

### REFERENCE TO SEQUENCE LISTING SUBMITTED VIA EFS-WEB

This application includes an electronically submitted sequence listing in .txt format. The .txt file contains a sequence listing entitled “15003-462US2_ST25.txt” created on Mar. 2, 2022 and is 3,298 bytes in size. The sequence listing contained in this .txt file is part of the specification and is hereby incorporated by reference herein in its entirety.

### BACKGROUND

### 1. Field of the Invention

The invention relates to the field of medicine and in particular to the field of cancer and cancer treatment. The invention provides a therapeutic diet that leads to tumor-specific decreases in glutathione levels, creating a pro-ferroptotic environment in tumors.

### 2. Background of the Invention

Each year an estimated 1.7 million people are diagnosed with cancer in the United States with 600,000 patients succumbing to cancer each year (American Cancer Society, 2017 figures). Glioblastoma is the most common malignant primary brain tumor, and has a median survival of only 14 months. Glioma treatment resistance has been linked to oxidative stress and glutathione metabolism. Oxidative stress, broadly defined as the (im)balance between reactive oxygen species and antioxidant defenses, underlies various distinct forms of cell death.

Ferroptosis is a form of regulated cell death that is iron dependent and mediated by lipid peroxidation. Glutathione, a reducing tripeptide with a thiol-containing cysteine residue, serves as a cofactor for the enzyme glutathione peroxidase 4 (GPX4) to donate electrons to peroxides of polyunsaturated fatty acyl phospholipids. Importantly, glutathione biosynthesis is dependent upon intracellular cysteine imported via the glutamate-cystine antiporter (system Xc⁻) and the enzymatic conversion of cysteine to glutathione. Methionine can also be converted to cysteine via the trans-sulfuration pathway to replenish glutathione. Therefore, ferroptosis inducers include compounds that inhibit system Xc⁻ (erastin, imidazole ketone erastin (IKE), sulfasalazine), compounds that directly inhibit GPX4 (RSL3, ML-210), and compounds that inhibit glutathione synthesis (buthionine sulfoximine).

There are more than 100 distinct types of cancers that share common hallmarks, including sustained proliferative signaling and evasion of growth suppressors. Cancers show diverse metabolic requirements influenced by factors such as tissue of origin, microenvironment, and genetics. The consumption profiles of cancer cells indicate homogeneous demands of energy metabolism and protein synthesis, which are vital biological processes for the malignant proliferation of cancer cells. The leading substrates consumed by cancer cells include glucose and amino acids, such as tryptophan, tyrosine, phenylalanine, lysine, valine, methionine, serine, threonine, isoleucine, leucine, and glutamine.

Additionally, cancer cells have increased iron demand and are more vulnerable to iron-catalyzed necrosis or ferroptosis. Cachexia is a multifactorial syndrome affecting many cancer patients that is associated with increased mortality and impaired response to chemotherapy. Dietary approaches to cancer treatment and cachexia may potentially improve treatment outcomes. There is a growing interest in targeting the metabolic environment in cancer and neurologic diseases. Currently no convenient solution for patients exists that enhance ferroptosis and/or deplete cysteine and methionine. Patient compliance is also a critical component for successful implementation, and can be a difficult problem for patients.

Providing a convenient mechanism for patients to be able to adhere to a restrictive diet prior to treatment is a critical need not addressed in the field. Therefore, there is a need in the art for a specific dietary regimen or formulation for cancer patients.

### SUMMARY OF THE INVENTION

Embodiments of the invention provide a dietary formulation and dietary compositions, as well as methods of treatment. The dietary formulations preferably contain sufficient calories and nutrients, particularly suitable macronutrients, for a complete diet for an adult human, and restrict intake of cysteine and methionine. In particular, the invention relates to a method of treating cancer in a subject in need thereof, the method comprising administering a cysteine and methionine deprivation (CMD) diet to the cancer subject.

In some embodiments, the CMD diet comprises a CMD formulation. In certain embodiments, the CMD formulation comprises (a) about 4% to about 60% fat by weight; (b) about 24% to about 73% carbohydrate by weight; (c) about 10% to about 25% protein by weight; (d) about 0% vitamin E by weight; (e) about 0% cysteine by weight; (f) about 0% to about 0.15% methionine by weight; (g) about 0% selenium by weight; (h) about 0% to about 10% saturated fatty acids by weight; (i) about 18 mg to about 65 mg iron per daily serving; and (j) about 0 g to about 50 g alanyl-glutamine per daily serving, wherein the poly unsaturated fatty acid (PUFA) to monounsaturated fatty acid (MUFA) ratio is at least 2:1.

In some embodiments, the CMD formulation comprises about 25% fat, about 53% carbohydrate, about 15% protein, and about 40 g per daily serving alanyl-glutamine.

In some embodiments, the methods above can further comprise co-administering radiotherapy to the subject in need. Preferably, the CMD diet and radiotherapy synergistically kill cancer cells, or the CMD diet reduces the coefficient of drug interaction of a given radiation dose of the radiotherapy.

In some embodiments, the CMD diet promotes iron ferroptosis in cancer cells in the subject.

In some embodiments, the methods described herein further comprise co-administering to the subject a chemotherapeutic agent. Preferably, the chemotherapeutic agent promotes iron ferroptosis in cancer cells in the subject.

In some embodiments, the invention also relates to a dietary formulation to reduce cysteine and/or methionine in a subject in need, comprising (a) about 4% to about 60% fat by weight; (b) about 24% to about 73% carbohydrate by weight; (c) about 10% to about 25% protein by weight; (d) about 0% vitamin E by weight; (e) about 0% cysteine by weight; (f) about 0% to about 0.15% methionine by weight; (g) about 0% selenium by weight; (h) about 0% to about 10% saturated fatty acids by weight; (i) about 18 mg to about 65 mg iron per daily serving; and (j) about 0 g to about 50 g alanyl-glutamine per daily serving, wherein the poly unsaturated fatty acid (PUFA) to monounsaturated fatty acid (MUFA) ratio is at least 2:1.

In particular embodiments, the dietary formulation comprises about 25% fat, about 53% carbohydrate, about 15% protein, and about 40 g per daily serving alanyl-glutamine.

Certain embodiments of the invention are dietary formulations in the form of a food product for oral consumption.

In addition the invention relates to embodiments which are articles of manufacture comprising a container and the dietary formulations described herein disposed therein.

### BRIEF SUMMARY OF THE DRAWINGS

FIG. 1 which provides 384-well close-response curves showing response to RSL3 from 6 glioma cell lines.

FIG. 2 is a set of photographs of live cell confocal microscopy of Bodipy-C11 labeled MG1 cells treated with 500 nM RSL3.

FIG. 3 is a set of photographs of live cell confocal microscopy of Bodipy-C11 labeled MG1 cells with 500 nM RSL3 and 2 uM ferrostatin-1.

FIG. 4 provides data on the representative close-response of MG1 cells treated with RSL3 (squares), RSL3 plus Ferrostatin-1 (light triangles), RSL3 plus 5 uM ZVAD-FMK (dark triangles), RSL3 plus 2 uM Nec-1s (inverted triangles).

FIG. 5 shows representative 384-well close-response curves for MG3 cells treated with RSL3 (dark triangles), RSL3 plus 2 uM Ferrostatin-1 (squares), CMD plus RSL3 (inverted triangles), CMD plus RSL3 and 2 uM Ferrostatin-1 (light triangles).

FIG. 6 is a graphs showing representative close-response curves of MG3 cell responses to ML-210 (dark triangles), ML-210 plus 2 uM Ferrostatin-1 (squares), CMD+ML-210 (inverted triangles), CMD+ML-210+2 uM ferrostatin-1 (light triangles).

FIG. 7 presents AUC quantification for close response curves from 3-independent 96-well close response curves of MG3 murine glioma cell lines treated with RSL3±CMD±2 μM Ferrostatin-1.

FIG. 8A shows representative close-response curves for MG1 glioma cells treated with RSL3±CMD±2 uM Ferrostatin-1.

FIG. 8B shows the AUC quantification for close-response curves from three murine glioma cell lines treated with RSL3±CMD±2 μM ferrostatin-1.

FIG. 8C shows AUC quantification for close response curves three human glioma cell lines treated with RSL3±CMD±2 μM ferrostatin-1.

FIG. 8D is a quantitation of 3 independent flow cytometry experiments using Bodipy-C11 for two additional murine glioma cell lines (MG2, MG3).

FIG. 9A, FIG. 9B, and FIG. 9C present representative Bodipy-C11 flow data from MG1 cells. FIG. 9A shows DMSO control (red), 100 nM RSL3 (blue), and 100 nM RSL3 plus 2 uM Ferrostatin-1 (orange) treatment for 30 minutes. FIG. 9B shows the same conditions but with 6 hours of cysteine methionine deprivation pretreatment. FIG. 9C shows a higher dose of RSL3 treatment (500 nM).

FIG. 9D and FIG. 9E are the quantitation of 3 independent experiments demonstrated in FIG. 9A through FIG. 9C.

FIG. 10A through FIG. 10D presents RT-qPCR data for CHAC1 (FIG. 10A), PTGS2 (FIG. 10B), SLC7a11 (FIG. 10C), and ATF4 (FIG. 10D) transcripts from MG1 cells in either control (black) or 24 hour CMD (grey) conditions.

FIG. 11A through FIG. 11C shows RT-qPCR data for TS543 cells after 48 hours CMD (grey) compared to control (black) for CHAC1 (FIG. 11A), SLC7A11 (FIG. 11B), and ATF4 (FIG. 11C) transcripts.

FIG. 12A through FIG. 12C show RT-qPCR data of ex vivo organotypic slices for CUMC Tumor Bank 6229 post-treatment recurrent glioblastoma treated in control (black) or CMD (gray) media. Transcripts for CHAC1 (FIG. 12A), SLC7a11 (FIG. 12B), and SLC7a11 (FIG. 12C) are shown.

FIG. 13A through FIG. 13C show the same data as FIG. B2C-12, using a high-grade R132H IDH1 mutated glioma. FIG. 13 shows RT-qPCR data of ex vivo organotypic slices for high-grade R132H mutant glioma, CUMC Tumor Bank 6234 ex-vivo organotypic slices in control or CMD media. Transcripts for CHAC1 (FIG. 13A), SLC7a11 (FIG. 13B, and ATF4 (FIG. 13C) are shown.

FIG. 14A is a principal component analysis of targeted metabolite profiling showing clustering along treatment conditions (light grey=control, dark grey=CMD).

FIG. 14B shows a pathway analysis of targeted metabolite profiling across control and CMD samples spanning 200 metabolites.

FIG. 14C is a heatmap showing top 50 differentially assessed metabolites based on FDR-corrected p-value, all <0.05.

FIG. 14D is a calorimetric assay of reduced glutathione levels for (left to right) MG1, MG2, MG3, TS543, and KNS42 in control (black bars) and CMD treated cells after 24 hours (gray bars).

FIG. 14Ei through FIG. 14Eiv shows the normalized metabolite concentrations for key metabolites, ascorbic acid, N-acetylputrescine, L-kynurenine, and deoxyuridine upregulated in CMD versus control, all with FDR<0.05.

FIG. 14Fi through FIG. 14Fviii shows the normalized metabolite concentrations for key metabolites, L-methionine, S-adenosylmethionine, L-cystine, and L-cystathionine downregulated in CMD versus control, all with FDR<0.05.

FIG. 14G presents data for basal oxygen consumption followed by sequential measurements of ATP-production (oligomycin inhibition), maximal respiration (FCCP inhibition) and mitochondrial respiration (rotenone/antimycin inhibition).

FIG. 14Hi, FIG. 14Hii, FIG. 14iii, and FIG. 14iv show the basal respiration, maximal respiration, ATP-linked respiration and proton leak values calculated from the experiment in FIG. 14I were calculated and normalized (n=5 per group).

FIG. 14I presents data on the extracellular acidification rate for control (black) or 12 hour CMD (gray) is shown.

FIG. 15A is diagram of the experimental paradigm.

FIG. 15B is a Kaplan-Meier curve outlining survival comparing control versus CMD diet mice orthotopically injected with MG3 cells

FIG. 16 shows the weights from C57/B6 male mice put on control or CMD diet.

FIG. 17 is a dot plot of the top 20 suppressed/activated protein/gene sets based on untargeted protein level enrichment analysis of FFPE end-stage samples from control (n=3) and CMD (n=4) male mice.

FIG. 18A and FIG. 18B are a Volcano plot and a heatmap, respectively, showing the top 50 differentially assessed metabolites) demonstrate the top differentially assessed metabolites.

FIG. 18C shows the correlation between oxidized glutathione and associated metabolites.

FIG. 18D is a schematic of cysteine metabolism with key differentially assessed metabolites with Log2FC and t-test p-value listed between control and CMD diet mice.

FIG. 19A presents a pathway analysis of targeted metabolite profiling across control and CMD male mice spanning 200 metabolites with relative concentrations log transformed and samples scaled by mean.

FIG. 19B shows a joint pathway analysis combining proteomics data of differential expression analysis comparing CMD vs. control and metabolite differential assessment analysis comparing CMD vs. control.

FIG. 19C shows representative DESI-MS images from tumor region overlay included for upregulated lipid species.

FIG. 19D shows representative DESI-MS images from tumor region overlaid included for downregulated lipid species.

FIG. 19E is a variable importance of projection diagram, showing lipid species important in discriminating the two classes of samples apart (FDR-corrected p-value <0.05) from 6 male mice (control n=3, CMD n=3) with data from negative ion mode shown,

FIG. 20A shows the results of in vitro experiment demonstrating that growing cells in CMD lead to lower levels of GSH.

FIG. 20B shows representative Bodipy-C11 flow data from MG1 cells.

FIG. 20C presents the quantitation of 3 independent flow cytometry experiments.

FIG. 20D presents close response curves showing the effects of IKE (48 hours) on mouse glioma cell viability in different media conditions.

FIG. 20E contains AUC analysis of close response for 2 mouse glioma cell lines.

FIG. 21A shows the quantitation of cell viability 120 hours after treatment with either control, CMD alone, 8 Gy irradiation alone, or CMD plus 8 Gy irradiation.

FIG. 21B shows the coefficient of drug interaction (CDI) quantitation for the cell viability data (CDI=AB/A×B), with CDI<1.0 indicating synergy between CMD and radiation.

FIG. 21C presents representative Bodipy-C11 flow cytometry data from MG4 cells showing increased lipid peroxidation with co-treatment of radiation plus CMD and complete rescue with Ferrostatin-1.

FIG. 21D shows the quantitation of 3 independent experiments of Bodipy-C11 lipid peroxidation in MG1, MG4 cells.

FIG. 21E shows quantitation of cell viability following 72 hours of treatment across 2 radiation closes and 4 conditions.

FIG. 21F presents the coefficient of drug interaction quantification for the cell viability data presented in FIG. 21E.

FIG. 22A and FIG. 22B provide data showing that CMD treatment in combination with radiation results in decreased rate of tumor growth in vivo as determined by luciferase imaging measuring tumor volume.

FIG. 23A and FIG. 23B provides data showing that CMD and radiation combined with temozolomide enhance tumor killing in vitro.

FIG. 24A provides data showing that CMD and radiation improve survival in vivo in a high grade mouse glioma model.

FIG. 24B is a schematic.

FIG. 25A provides the same data as FIG. 24A, for a low grade glioma model.

FIG. 25B is a schematic.

FIG. 26A and FIG. 26B are representative histograms showing a human diffuse astrocytoma slice culture sample treated with DMSO, 10 μM IKE, or 10 μM IKE+10 μM ferrostatin-1, and co-treated with 0 or 2 Gy radiation for 24 hours.

FIG. 26C shows the H2DCFDA staining of three human glioma slice culture samples treated with same conditions.

FIG. 27A is a set of confocal images of tissue slices stained with propidium iodide.

FIG. 27B presents data from 4 random areas of each slice, quantitated for mean fluorescence intensity.

FIG. 28 is a bar graph showing that altering cysteine and methionine concentrations alters tumor viability and susceptibility to ferroptosis in vitro.

FIG. 29 is a graph showing the response of glioma cells to cysteine/methionine deprivation and radiation.

FIG. 30 is a graph showing the response of glioma cells to cysteine/methionine deprivation and RSL-3 chemotherapy.

FIG. 31 is a graph showing that glioma growth is slowed in vivo when mice are placed on a cysteine deprived/methionine restricted diet.

FIG. 32 is a graph showing that a cysteine deprived/methionine restricted diet improved survival in a mouse model of diffusely infiltrating glioma.

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2. In reference to zero, such as “about 0%,” the term “about” refers to an undetectable amount and includes zero, or none.

As used herein, the term “ferroptosis” refers to a type of cell death that differs from traditional apoptosis and necrosis and results from iron-dependent lipid peroxide accumulation. Ferroptotic cell death is characterized by cytological changes, including cell volume shrinkage and increased mitochondrial membrane density.

As used herein, the term “cancer therapy” refers to a therapy, such as surgery, chemotherapy, radiotherapy, thermotherapy, and laser therapy administered to a cancer patient.

As used herein, the term “cancer therapeutic agent” pertains to an agent that possesses selectively cytotoxic or cytostatic effects to cancer cells over normal cells. Adjunct cancer therapeutic agents may be co-administered with a CMD diet or formulation, and optionally in further combination with radiotherapy. An example of a cancer therapeutic agent that promotes ferroptosis includes, but is not limited to RSL3. Ferroptosis-inducing drug RSL3 selectively targets mesenchymal glioma cells.

As used herein, the term “radiotherapy” or “radiation therapy” refers to administration of beams of intense energy to kill cancer cells in a subject. Radiation therapy most often uses X-rays, but protons or other types of energy also can be used. In a particular example, the radiotherapy induces ferroptosis selectively in cancer cells. Radiotherapy may be co-administered with a CMD diet or formulation such that the CMD diet or formulation increases effectiveness (increased cytotoxicity per dose) of the radiotherapy. In a more specific example, the CMD diet or formulation synergistically induces ferroptosis with radiotherapy.

As used herein, the term “subject” refers to a human or non-human animal, for example humans, laboratory animals (e.g., rats, mice, rabbits, and the like), companion animals (e.g., cats dogs, and the like), farm animals (e.g., horses, cattle, sheep, and the like), zoo animals, or any animal in need. A preferred subject is human.

As used herein, the term “cysteine methionine deprivation (CMD)” diet refers to a diet that intentionally deprives or restricts a subject of cysteine and/or methionine while also satiating the subject.

As used herein, the term “CMD formulation” refers to a combination of ingredients in a composition designed for consumption by a subject, wherein the CMD formulation lacks cysteine and methionine and whose consumption provides an example of a CMD diet. Examples of a CMD formulation may be a powder, shake, drink, nutritional bar or other food product. The CMD formulation meets the Essential constituent criteria provided above.

As used herein, the term “cysteine deprived-methionine restricted (CDMR)” diet refers to a diet or dietary formulation that contains substantially no cysteine (no added cysteine or not detectable cysteine) and also a very low concentration (restricted concentration) of methionine, The term “deprived” is used herein to mean zero. CDMR is encompassed by the broad term CMD.

As used herein, the term “therapy” as used herein includes CMD diet, CMD formulation, or cancer therapy (e.g. radiotherapy or chemotherapy).

As used herein, the term “administering” and its cognates refer to introducing or providing a therapy to a subject, and when the therapy is an agent, formulation or CMD diet, can be performed using any of the various methods or delivery systems for administering agents or pharmaceutical compositions, and any route suitable for the composition and the subject, as known to those skilled in the art. Modes of administering include, but are not limited to oral administration, intravenous, subcutaneous, intramuscular or intraperitoneal injections, or local administration directly into or onto a target tissue (such as the pancreas, brain, or a tumor). Administration by any route or method that delivers a therapeutically effective amount of the drug or composition, or other type of therapy, to the cells or tissue to which it is targeted is suitable for use with the invention.

As used herein, the term “co-administration” or “co-administering” refers to the administration of a therapy (e.g. CMD diet), concurrently, or after the administration of another therapy (e.g. radiation therapy or chemotherapy) such that the biological effects of either therapy overlap. The combination of therapies as taught herein can act synergistically to treat or prevent the various diseases, disorders or conditions described herein. Using this approach, one may be able to achieve therapeutic efficacy with lower dosages of each agent, thus reducing the potential for adverse side effects.

As used herein, the term “ferroptosis” refers to an iron dependent form of regulated cell death mediated by lipid peroxides. Ferroptosis results from iron-dependent lipid peroxide accumulation and is characterized by cytological changes, including cell volume shrinkage and increased mitochondrial membrane density.

As used herein, the term “therapeutically effective amount” refers to an amount sufficient to treat a subject in need as described below. Preferably, this amount is sufficient to induce tumor killing, halt or reduce tumor growth, enhance the tumor killing ability of other agents, or enhance the ability of other agents to halt growth as determined by direct measurements or surrogates such as clinical or radiographic progression of disease, or survival.

As used herein, the terms “treatment,” “treating,” and the like, refer to obtaining a desired pharmacologic and/or physiologic effect through administering a therapy, agent or formulation. The effect may be prophylactic in terms of completely or partially preventing a condition or disease or symptom thereof and/or may be therapeutic in terms of a partial or complete cure for a condition or disease and/or adverse effect attributable to the condition or disease. “Treatment” includes: preventing, partially preventing, reversing, alleviating, reducing the likelihood of, or inhibiting the condition or disease (or symptom thereof) from occurring in a subject. The subject can include those diagnosed with a tumor or cancer, a pre-cancer, or who are predisposed to the condition or disease but has not yet been diagnosed as having it; (b) inhibiting the condition or disease or symptom thereof, such as, arresting its development; and (c) relieving, alleviating or ameliorating the condition or disease or symptom thereof, such as, for example, causing regression of the condition or disease or symptom thereof. Treatment can include administering one or more agents, performing a procedure such as surgery or applying radiation and the like, or both.

As used herein, the term “coefficient of drug interaction (CDI)” refers to E_{AB}/(E_{A}*E_{B}); where E_{A}=Effect of drug A; E_{B}=Effect of Drug B; E_{AB}=Effect of co-administration of drugs A+B.

### 2. Overview

A broad range of cancer cell lines, including glioma, are sensitive to ferroptosis inducers. Moreover, compounds that target system Xc⁻ can synergize with radiation to increase reactive oxygen species (ROS) generation and lipid peroxidation in ex vivo organotypic glioma slices. Given the centrality of glutathione to protect from ferroptosis, depletion of its precursors, cysteine and methionine, should sensitize cells to undergo ferroptosis. Pharmacologic means of cysteine deprivation have been shown to be efficacious in other cancers. However, blood-brain barrier penetration remains an obstacle for any central nervous system target. Therefore, to determine the effect of dietary restriction of cysteine and methionine on glioma the studies described here were performed. Cell death, lipid peroxide generation, and transcriptional hallmarks of ferroptosis are enhanced by cysteine and methionine deprivation (CMD).

The dietary formulation and methods described herein have the ability to improve a broad range of treatments. The data presented herein concerning radiation is important for cancer treatment because up to 50% of cancer patients will receive radiation, many needing multiple rounds. The ability for this diet to improve a broad range of treatments, specifically radiation treatments, shows its immense utility in improving clinical care for this patient population.

### 3. Summary of Results

Our analysis of the effects of CMD in vitro showed significant decreases in metabolites in 3 major pathways including cysteine-methionine metabolism, taurine/hypotaurine metabolism, and glutathione synthesis. These findings translated to the in vivo setting where an orthotopic mouse glioma model treated with CMD diet showed decreases in pathways related to glutathione synthesis, and hypotaurine/taurine metabolism. This demonstrates that the systemic dietary deprivation provided by the invention affects tumor metabolism and growth within the central nervous system.

Aerobic and anaerobic respiration were decreased following CMD in vitro, Also, in vivo metabolomic data showed alteration within pyruvate and TCA cycle pathways, particularly acetyl-CoA was negatively correlated with oxidized glutathione levels and may demonstrate a cellular escape mechanism to chronic CMD exposure.

The CMD diet was shown to be a ferroptotic stress as demonstrated through a multiomic approach. DESI-IMS data showed a shift in tumor lipid profiles towards more pro-ferroptotic species. The levels of PI 38:4 and PS 40:6 (PI 18:0_20:4, PS 18:22:6), phospholipids with PUFA tails, were increased significantly in the CMD group. Moreover, phospholipids with saturated and monounsaturated fatty acid tails are ferroptosis resistant. PC 16:0_18:1, one of the most abundant phospholipids in the brain with proven anti-ferroptotic activity, was depleted significantly in the CMD group.

Notably, upregulation of FA18:2, a omega-6 PUFA tied to decreased antioxidant capacity was seen in vivo. Here the inventive CMD diet was shown to be a non-toxic, chronically tolerated regimen associated with a modest but significant survival benefit, indicating local effects on brain tumor growth from a systemic diet. The CMD diet was also associated with key tumor specific metabolic and lipid changes that are promising avenues for future investigation and combination treatment.

In summary, this CMD diet leads to tumor-specific decreases in glutathione levels in vitro. In addition, a methionine-restricted cysteine-depleted diet is safe in vivo and decreases glutathione levels in vivo. Finally, this in vivo dietary paradigm improves survival in an orthotopic syngeneic murine model of glioma and alters the lipid composition of tumors to create a pro-ferroptotic environment. These results support using CMD diet as a non-invasive method for improving the efficacy of ferroptotic treatments and survival of glioma patients.

### 4. Embodiments of the Invention

### Formulations

Murine and human glioma cells are susceptible to ferroptosis via GPX4 inhibition by drugs such as RSL3. RSL3-mediated cell death is ferroptosis-specific (independent of apoptosis or necroptosis) and is associated with increased lipid peroxidation. Moreover, nutrient deprivation of cysteine and methionine decreases cancer cell survival, and synergistically increases lipid peroxidation and cell death when combined with RSL3. Ex vivo slices from human gliomas showed both synergistic sensitivity to CMD and ferroptosis inducers as well as significant transcriptional upregulation of CHAC1 and SLC7a11 following CMD. In vivo dietary deprivation of cysteine and methionine resulted in increased survival with distinctive changes in the lipidomics, proteomics and metabolomic profile of the tumors.

The dietary formulation and supplement are specifically designed to maximize ferroptosis by depleting cysteine and methionine, in addition to altering the proportions of key ingredients demonstrated to enhance efficacy of cancer treatments. The dietary formulation and supplement also target cancer patients with and adult patients that have adequately higher energetic needs than currently available supplements provide. By restricting sulfur-containing amino acids from the patient's diet, cancer cells can be selectively targeted and sensitized to treatment. Therefore, the dietary formulations provide dual restriction of the sulfur-containing amino acids methionine and cysteine as a dietary intervention to selectively target cancer cells, sensitizing them to radiation and standard-of-care cancer chemotherapies.

The normal diet of a patient (which would contain cysteine and methionine) can interfere with the benefits of providing the CMD diet. Thus, the formulation embodiments preferably provide enough caloric content and fat to sufficiently satiate the patient so that the appetite is satisfied to avoid the patient consuming foods that could disrupt the benefits of the CMD diet. Thus, the formulation contains a satisfying amount of fat, carbohydrates and protein.

The dual deprivation of sulfur-containing amino acids (methionine and cysteine) is able to selectively target cancer cells and sensitize them to both radiation and standard of care chemotherapies. While the diet and formulation embodiments are particularly adaptable for treating glioma/glioblastoma, the mechanism for how the formulation and related diet works is broadly applicable to other cancer types where patients commonly undergo radiation or chemotherapy treatments. Adhering to this diet can greatly improve patient outcomes.

In certain embodiments, the invention provides a specific restricted dietary formulation and methods for the treatment of various cancers. In one embodiment, embodiments based on the diet are formulated as a nutritional powder or granules or a liquid or semi-liquid or slurry/shake with a defined caloric intake that intentionally excludes two amino acids: cysteine and methionine. The dietary formulation thus can be formulated with additional inert or non-active ingredients, carriers, or fillers. Such inert ingredients can include water, electrolytes, suspending agents, gelling agents, thickeners, soluble and/or insoluble fiber, inert fillers, flavorings, and the like.

Preferably, the dietary formulations are created for adult patients taking into account adult energetic needs, but they can be modified for pediatric patients. The formulations also preferably are designed to contain a high enough caloric density to provide to the specific cancer patient population sufficient calories and nutrition, since cancer patients commonly have energetic needs that are more difficult to meet and require high caloric density to continue through the rigors of chemotherapy and radiation treatment. Therefore, certain embodiments of the dietary formulation contains about 0 to about 4 kcal per serving, preferably about 1,250 to about 2,500 kcal per serving and most preferably about 1,500 to about 2,500 kcal. The serving of the formulations according to the embodiment can be determined by the treating physician, oncologist, or nutritionist based on the patient's size and weight, general health, severity of disease, activity level, caloric need, nutritional status, and the like.

In one embodiment, a dietary formulation according to the invention comprises a combination of constituents that when administered to a cancer patient promotes ferroptosis and/or increases selectively toxicity of cancer cells to a cancer therapy. Thus, the formulation includes no or only basal methionine and no cysteine in order to enhance ferroptosis (i.e., iron-mediated lipid peroxidation).

In specific embodiments, the formulation comprises the substances in Table 1, below, and optionally also can contain one or more of: Vitamin A (0-900 mcg retinol activity equivalents (RAE)), Vitamin C (0-90 mg), Vitamin D (0-20 mcg), Vitamin K (0-120 mcg), Thiamin (0-1.2 mg), Riboflavin (0-1.3 mg), Niacin (16 mg of niacin equivalents (NE)), Vitamin B6 (0-1.7 mg), Folate (0-400 mcg dietary folate equivalents (DFE)), Vitamin B12 (0-2.4 mcg), Biotin (0-30 mcg), Pantothenic Acid (0-5 mg), Choline (0-550 mg), Calcium (0-1300 mg), Phosphorus (0-1250 mg), Iodine (0-150 mcg), Magnesium (0-420 mg), Zinc (0-11 mg), Copper (0-0.9 mg), Boron (0-13 mg). Table 1 refers to an example 14 oz or 24 oz serving by weight, which contains about 1,250 kcal or 2,500 kcal, respectively. Units are per serving by weight for a solid powder formulation before addition of water or other diluent. Other compositions (e.g. capsules, packets, pouchs, tablets, and the like would have equivalent formulations.

TABLE 1
Example Dietary Formulation Components.

| Essential | | | More | Most |
|---|---|---|---|---|
| Component | Range | Preferred | Preferred | Preferred |
| Fat* | About 4%- | About 10%- | About 20%- | About 25% |
| | about 60% | about 50% | about 40% | |
| Carbohydrate | About 24%- | About 30%- | About 40%- | About 53% |
| | about 73% | about 70% | about 60% | |
| Protein | About 10%- | About 12%- | About 13%- | About 15% |
| | about 20% | about 18% | about 17% | |
| Vitamin E | About 0% | | | About 0% |
| Selenium | About 0% | | | About 0% |
| Iron | About 18 g- | About 20 g- | About 30 g- | About 40 g- |
| | about 65 g | about 60 g | about 50 g | about 45 g |
| Alanyl- | About 0 g- | About 5 g- | About 20 g- | About 40 g |
| glutamine | about 50 g | about 30 g | about 35 g | |
| Cysteine | About 0% | | | About 0% |
| Methionine | About 0%- | | | About 0% |
| | about 0.15% | | | |

*polyunstaturated fatty acid (PUFA):monounsaturated fatty acid (MUFA) ratio at least 2:1 or greater, and saturated fatty acid (SFA) < about 10% (range 0-10%).

Table 2, below, provides several specific examples of dietary formulations according to the invention. Units are per serving unless noted otherwise.

TABLE 2
Example Formulations.

| Component | Formula A | | Formula B | | Formula C | | Formula D | | Formula E | | Formula F | |
|---|---|---|---|---|---|---|---|---|---|---|---|---|
| PUFA | 2:1of | | 2:1of | | 2:1of | | 5:1of | | 10:1of | | 20:1of | |
| | total fat | | total fat | | total fat | | total fat | | total fat | | total fat | |
| MUFA | 1:2 of | | 1:2 of | | 1:2 of | | 1:5 of | | 1:10 of | | 1:20 of | |
| | total fat | | total fat | | total fat | | total fat | | total fat | | total fat | |
| SFA | 5% of | | 5% of | | 5% of | | 1% of | | 1% of | | 1% of | |
| | total fat | | total fat | | total fat | | total fat | | total fat | | total fat | |
| Total Fat | 25% | | 30% | | 30% | | 25% | | 30% | | 30% | |
| Carbohydrate | 53% | | 57% | | 57% | | 53% | | 57% | | 57% | |
| Protein | 15% | | 13% | | 13% | | 15% | | 13% | | 13% | |
| Iron | 65 | mg | 65 | mg | 30 | mg | 65 | mg | 65 | mg | 65 | mg |
| Alanyl- | 40 | g | 40 | g | 40 | g | 40 | g | 40 | g | 40 | g |
| glutamine | | | | | | | | | | | | |
| Vitamin A | 900 | mcg | 900 | mcg | 900 | mcg | 900 | mcg | 900 | mcg | 900 | mcg |
| Vitamin C | 90 | mg | 90 | mg | 90 | mg | 90 | mg | 90 | mg | 90 | mg |
| Vitamin D | 20 | mcg | 20 | mcg | 20 | mcg | 20 | mcg | 20 | mcg | 20 | mcg |
| Vitamin K | 120 | mcg | 120 | mcg | 120 | mcg | 120 | mcg | 120 | mcg | 120 | mcg |
| Vitamin B6 | 1.2 | mg | 1.2 | mg | 1.2 | mg | 1.2 | mg | 1.2 | mg | 1.2 | mg |
| Vitamin B12 | 1.3 | mg | 1.3 | mg | 1.3 | mg | 1.3 | mg | 1.3 | mg | 1.3 | mg |
| Riboflavin | 1.3 | mg | 1.3 | mg | 1.3 | mg | 1.3 | mg | 1.3 | mg | 1.3 | mg |
| Niacin | 16 | mg NE | 16 | mg NE | 16 | mg NE | 16 | mg NE | 16 | mg NE | 16 | mg NE |
| Folate | 400 | mcg NFE | 400 | mcg NFE | 400 | mcg NFE | 400 | mcg NFE | 400 | mcg NFE | 400 | mcg NFE |
| Biotin | 30 | mcg | 30 | mcg | 30 | mcg | 30 | mcg | 30 | mcg | 30 | mcg |
| Pantothenic | 5 | mg | 5 | mg | 5 | mg | 5 | mg | 5 | mg | 5 | mg |
| Acid | | | | | | | | | | | | |
| Choline | 550 | mg | 550 | mg | 550 | mg | 550 | mg | 550 | mg | 550 | mg |
| Calcium | 1300 | mg | 1300 | mg | 1300 | mg | 1300 | mg | 1300 | mg | 1300 | mg |
| Phosphorus | 1250 | mg | 1250 | mg | 1250 | mg | 1250 | mg | 1250 | mg | 1250 | mg |
| Iodine | 150 | mcg | 150 | mcg | 150 | mcg | 150 | mcg | 150 | mcg | 150 | mcg |
| Magnesium | 420 | mg | 420 | mg | 420 | mg | 420 | mg | 420 | mg | 420 | mg |
| Zinc | 11 | mg | 11 | mg | 11 | mg | 11 | mg | 11 | mg | 11 | mg |
| Copper | 0.9 | mg | 0.9 | mg | 0.9 | mg | 0.9 | mg | 0.9 | mg | 0.9 | mg |
| Boron | 13 | mg | 13 | mg | 13 | mg | 13 | mg | 13 | mg | 13 | mg |

### Methods of Use

In some embodiments, the invention relates to methods for treating a subject in need, by providing the dietary formulations described herein as the only source of nutrition. The formulation is provided to the patient in a form that can be consumed as a liquid, or as a powder or granules that can be dissolved or made into a slurry by addition of water or some other diet-compatible liquid. The patient can consume the diet by mouth, by nasogastric tube, or any suitable method as designated by the practitioner. The dietary formulation can be provided in divided doses to provide sufficient nutritional and caloric needs for the patient.

This invention has been tested here on a notoriously hard-to-treat cancer: glioma/glioblastoma. Without wishing to be bound by theory, ferroptosis is induced by inhibition of GPX4, an enzyme that facilitates glutathione-mediated detoxification of toxic lipid peroxides, and therefore is a promising avenue for cancer treatment.

The clinical evidence presented here thus is broadly applicable and can easily be applied to other cancer types since patients commonly undergo radiation or chemotherapy treatments for a variety of cancers. This specific diet and dietary formulation is provided to or administered to patients, for example patients who have been diagnosed with cancer or pre-cancer, or who are suspected of having cancer. Preferably the diet is provided before cancer treatment begins and continues during treatment. The cancer treatment can be any standard-of-care treatment as determined by the practitioner of skill, however the preferred cancer treatments for use with methods according to the invention are radiation or chemotherapy with a focus on ferroptosis-inducing agents such as RSL3, Erastin, and the like. Other therapies contemplated for use with the inventive methods include surgery, laser ablation, focused ultrasound, and the like.

In addition to cancer treatment, the embodiments of the invention are contemplated for use in disease states or conditions that are treated or treatable using radiation. Such conditions include, but are not limited to: benign tumors, vascular malformations, neuralgia/chronic pain conditions, spinal cord tumors, spine disc herniations, and the like.

Other conditions and disease states also can be treated with the dietary formulation according to the invention. In some embodiments, the conditions are neurodegenerative disorders such as Alzheimer's disease, Parkinson's disease, other dementias, and patients at high risk for dementia. In other embodiments, the dietary formulation also can benefit patients with obesity and related illnesses (i.e. diabetes, hypertension) and be useful in treatments for cachexia, especially cachexia associated with cancer or HIV/AIDS. In addition, the dietary formulation can be used as a health supplement for malnutrition and in research models for studying amino acid metabolism. This dietary therapy has the potential to greatly improve outcomes for patients with diverse cancers and other metabolic and neurologic disorders.

Preferred subjects in need, with respect to embodiments of the invention include any subject that has been diagnosed with cancer or is suspected of having cancer, including glioma/glioblastoma or any cancer such as pancreatic cancer or colon cancer. Additional subjects in need include patients suffering from any condition that can be benefited by the methods and compositions described herein. Such subjects generally are patients suffering from a disease or condition selected from the group consisting of cancer, neurodegenerative disorders (e.g., Alzheimer's disease, Parkinson's disease, and the like), metabolic disorders (e.g., metabolic syndrome, type 2 diabetes, obesity, and the like), and benign tumors.

In some specific embodiments, a CMD diet or CMD diet formulation is administered to a cancer patient prior to and/or during standard-of-care cancer treatment for a sufficient time to diminish cancer cell resistance to the therapy. The standard-of-care cancer treatment can include surgery, chemotherapy, and/or radiation therapy, but in a preferred embodiment, the cancer therapy is radiation therapy or chemotherapy involving an agent that induces ferroptosis in the cancer patient, such as RSL3. The CMD diet or formulation and the cancer treatment preferably are provided in a therapeutic amount so as to induce a synergistic effect. In one embodiment, a synergistic effect is recognized when a coefficient of drug interaction (CDI) of <1.0. (CDI=AB/A×B). AB is the ratio of the combination groups to control group; A or B is the ratio of the single agent group to control group. Thus, CDI<1, =1 or >1 indicates that the drugs are synergistic, additive or antagonistic, respectively.

In other embodiments, the CMD dietary formulation is provided or administered to any subject as described herein before, after, during, conventional therapy, as determined by a medical practitioner, or a combination thereof.

In certain embodiments, a CMD diet or formulation is provided to a patient in a regimen (dosage, duration and frequency) so as to reduce a CDI for a given radiation close. Typically, the CMD regimen and radiation will be of an amount to achieve a CDI<1.0. In another embodiment, a CMD diet or formulation is provided to a patient in a regimen so as to reduce a CDI of a dose of a chemotherapeutic agent.

In another embodiment, provided is a method of treating cancer comprising co-administering a CMD diet or formulation and a radiation treatment, such that the CMD diet or formulation increases the effectiveness of the radiation treatment in killing cancer cells. Another embodiment, pertains to a method of treating cancer comprising co-administering a CMD diet or formulation and an amount of a chemotherapeutic agent such that the CMD diet or formulation increases effectiveness of the chemotherapeutic agent to kill cancer cells.

The dietary formulation is well-tolerated and can be used by cancer patients about to begin radiation and/or chemotherapy treatment to assist in selectively targeting and sensitizing cancer cells to radiation and chemotherapy. Administering the dietary formulation has been tested in an in vivo mouse model and shown to decrease glioma growth and increase survival without observable toxicity.

Preferred amounts and regimens for administration include a daily amount sufficient to meet the caloric and other nutritional needs of the patient, given in one dose or in divided doses throughout the day. A preferred amount for an average human is sufficient to supply about 2000 to about 2500 calories, about 2000 to about 4000 calories, or about 0 to about 4000 calories. In other embodiments, the patient can determine the amount of the dietary formulation to be consumed in order to provide satiety. Preferably, no other unapproved nutrition is taken by the patient while on the CMD diet in order to avoid consuming methionine or cysteine.

The CMD diet can be given for one day or for extended periods, including up to two years or indefinitely. Preferably, the diet is begun about 3 days to about 14 days prior to the standard-of-care therapy designed for the patient up until therapy begins, or continuing through therapy, and optionally beyond. The CMD diet is contemplated to continue for at least about 1 weeks to about 3 months, preferably about 1 weeks to about 2 months.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### Discussion

In vitro assays were performed using glioma cell lines previously established by our laboratory. Cell viability was assessed using bioluminescence. Flow cytometry was used to determine changes in lipid peroxidation (BODIPY-C11) and reactive oxygen species (ROS) (H2DCFDA) in normal and CMD conditions. In vivo studies were performed using stereotactic orthotopic injections in syngeneic mice, fed control or CMD diet. Subsets were also treated with radiation to assess synergy by measuring tumor burden via bioluminescence and survival.

### Cell Lines and Culture Conditions

Murine glioma cell lines were generated according to known methods described in the art. Briefly. C57Bl/6 mice harboring floxed p53 and stop-flox mCherry-luciferase were orthotopically injected with a PDGFA-internal ribosomal entry site (IRES)-cyclization recombination (Cre) retrovirus (stereotaxic coordinates relative to bregma: 2 mm anterior, 2 mm lateral, 2 mm deep), resulting in tumor cells that overexpress PDGFA and mCherry-Luciferase, and have deleted p53. End-stage tumors were harvested and tumor cells isolated and cultured in basal media (BFP), containing DMEM (Gibco™ 11965092) with 0.5% FBS (Gibco™ 16000044), antibiotic-antimycotic (Thermo Scientific™ 15240096). N2 supplement (Thermo Fisher Scientific™, 17502-048), and 10 ng/ml each of recombinant human PDGF-AA (Peprotech™, 100-13A) and FGFb (Peprotech™, 10018B50UG). Three biological replicates of PDGFA driven cells made from three independent tumors with the same genetic background were used for this study. A Pten^{-/-} P53^{-/-} PDGFB⁺ cell line was also used. Cells were grown at 37° C. with 5% C02. Cysteine methionine deprived media was made from basal DMEM without cysteine, methionine and glutamine (Thermo Fisher Scientific™. 21013024) that was supplemented with L-glutamine to a final concentration of 4 mM. Human glioma cells were cultured as previously described.

Generation of Acute Organotypic Slice Cultures from Mouse Brains and Human Surgical Specimens.

Mouse or human brain slice cultures were generated as described previously in the art. Mice were sacrificed by cervical dislocation. The brain was removed and placed into an ice-cold sucrose solution (210 mM sucrose, 10 mM glucose, 2.5 mM KCl, 1.25 mM NaH2PO4, 0.5 mM CaCl₂), 7 mM MgCl2 and 26 mM NaHCO₃). After 20 minutes, the brain was cut into 300-500 μm sections using a McIlwain™ Tissue Chopper. After cutting, slices were rested in the sucrose solution for 20 minutes, then transferred onto Millicell™ cell culture inserts (0.4 μM, 30 mm diameter) and placed in 6-well plates containing 1.5 mL of medium consisting of DMEM/F12 with N-2 Supplement and 1% antimycotic/antibiotic. Human surgical specimens were collected from Columbia University Medical Center operating theaters, deidentified and placed in a sterile 50 mL conical tube containing the ice-cold sucrose solution for transportation. For treatment conditions, Hams-F12 without cysteine or methionine (MyBioSource™, MBS652871) was mixed 1:1 with DMEM without cysteine or methionine (Thermo Fisher Scientific™, 21013024) to make the DMEM/F12 without cysteine/methionine.

### Real Time Quantitative Polymerase Chain Reaction Primers.

Primers were found using the Harvard qPCR Primer Bank. The primers sequences used are provided below in Table 3, below.

TABLE 3
RT-qPCR primers for transcriptional assays.

| | | SEQ |
|---|---|---|
| Primer | | ID |
| Transcript Name | Oligo Sequence (5′→3′) | NO |
| Human beta-Actin | CATGTACGTTGCTATCCAGGC | 1 |
| forward | | |
| Human beta-Actin | CTCCTTAATGTCACGACGAT | 2 |
| reverse | | |
| Human SLC7a11 forward | TCTCCAAAGGAGGTTACTGC | 3 |
| Human SLC7a11 reverse | AGACTCCCCTCAGTAAAGTGAC | 4 |
| Human ATF4 forward | ATGACCGAAATGAGCTTCCTG | 5 |
| Human ATF4 forward | GCTGGAGAACCCATGAGGT | 6 |
| Mouse beta-Actin | CGAGGCCCAGAGCAAGAGAG | 7 |
| forward | | |
| Mouse beta-Actin | CTCGTAGATGGGCACAGTGTG | 8 |
| reverse | | |
| Mouse ATF4 forward | CCTGAACAGCGAAGTGTTGG | 9 |
| Mouse ATF4 reverse | TGGAGAACCCATGAGGTTTCAA | 10 |
| Mouse SLC7a11 forward | GGCACCGTCATCGGATCAG | 11 |
| Mouse SLC7a11 reverse | CTCCACAGGCAGACCAGAAAA | 12 |
| Mouse PTGS2 forward | TTCAACACACTCTATCACTGGC | 13 |
| Mouse PTGS2 reverse | AGAAGCGTTTGCGGTACTCAT | 14 |
| Mouse/Human ChacI | CTGTGGATTTTCGGGTACGG | 15 |
| forward | | |
| Mouse/Human ChacI | CCCTATGGAAGGTGTCTCC | 16 |
| reverse | | |

Real time Quantitative PCR Method.

RNA was extracted using the RNeasy™ Mini kit (QIAGEN). For tissue lysis, a 5 mm stainless steel bead (QIAGEN) was used to facilitate tissue lysis prior to RNA extraction. Following RNA extraction, up to 2.5 μg of RNA was used with the SuperScript™ Vilo cDNA synthesis kit (ThermoFisher™). cDNA was diluted to a concentration of 250 ng/μL and the RT-qPCR reactions were conducted with Thermo Scientific™ ABsolute Blue qPCR SYBR (ThermoFisher™). Duplicate samples per condition were analyzed on an Applied Biosystems QuantStudio™ 3 qPCR instrument with all experiments being repeated 3 independent times. beta-Actin was used as reference and log fold change was calculated using the ddCT method comparing treatments to a control sample.

### Cell Viability Assays—RSL3.

Cell viability was assessed using the Cell-Titer^{Glo}™ luminescence assay. Murine glioma cells were plated in triplicate at a density of 6,000 cells per well in a 96-well plate (ThermoFisher Scientific™). Twenty-four hours after plating, media was removed and treatment media was added. Viability was assessed 24 hours after treatment. Human glioma cells were plated at a density of 2,000 cells per well. Cells were plated in normal media or cysteine/methionine deprived media. Twenty-four hours after plating, media was changed to begin drug treatment. Forty-eight hours after plating, luminescence was measured.

Averages across 3 independent experiments are reported. For experiments conducted in 384-well plates, mouse glioma cells were plated at a density of 1,600 cells per well and human glioma cells were plated at a density of 1,000 cells per well. The assays as described above were quantified using Cell-Titer Glo™ (Promega™) ATP based bioluminescence. To determine cell viability, a 50% Cell Titer Glo™ and 50% cell culture medium was added to each well and incubated at room temperature for 10 minutes. Luminescence was assessed on a Promega™ GloMax™ Microplate Reader.

### Flow Cytometric Analysis of Lipid Peroxidation or ROS Cell Lines.

Adherent cells were lifted using TrypLE (ThermoFisher™ Scientific). Cell pellets were resuspended in 1 mL PBS with either Bodipy-C11 (ThermoFisher™) or H2DCFDA (ThermoFisher™) were added to a final concentration of 2 μM and 5 μM respectively. Cells were incubated with the dyes for 10 minutes at 37° C. Cells were centrifuged at 400×g for 5 minutes then resuspended in PBS.

### Slice Cultures.

Slice cultures were dissociated in papain (9.5 mL DPBS, 500 μL papain, 1.67 μL 6 M NaOH, 2 mg L-cysteine, 100 μL DNAse) and incubated in a warm bath shaker at 37° C. for 30 minutes. After centrifugation at 400×g for 5 minutes, the papain was aspirated and slices were resuspended in ice cold PBS and triturated with glass tip Pasteur pipettes. This process was repeated one time and then the cell suspension was resuspended in a 30% sucrose solution and spun at 1000×g for 5 minutes. The cell suspension was resuspended in PBS and stained with Calcein Blue (final concentration 5 μM) and H2DCFDA (final concentration 10 μM), incubated in a water bath at 37° C. for 10 minutes. Suspensions were spun down at 500×g for 5 minutes and resuspended in PBS and taken for flow cytometric analysis on a LSRIII Fortessa™ machine. Cell and Slice culture suspensions were filtered in polystyrene flow tubes (Fisher Scientific™). Data were collected on an LSRIII Fortessa™ flow analyzer and analyzed using FlowJo™ v10.

### Time Lapse Confocal Imaging of Lipid Peroxidation.

1.3×10⁵ MG1 cells were plated on poly-L-lysine coated 35 mm glass-bottom dishes (MatTek™ life sciences) for 24 hours. Cells were incubated for 30 minutes in basal media (DMEM (Gibco™ 11965092) with 0.5% FBS (Gibco™ 16000044), antibiotic-antimycotic (Thermo Scientific™ 15240096), N2 supplement (Thermo Fisher Scientific™, 17502-048), and 10 ng/ml each of recombinant human PDGF-AA (Peprotech, 100-13A) and FGFb (Peprotech™. 10018B50UG)) media containing 5 μM BODIPY-C11. Media were replaced with fresh basal media and cells were imaged on a Nikon™ A1RMP confocal microscope at 37° C. in a humidified chamber with 5% C02. Time-lapse images were acquired using a 40×/1.3 NA oil immersion objective and focus was maintained using the Perfect Focus™ System. Excitation was achieved using 488 nm and 561 nm laser illumination; emission of the oxidized and reduced forms of BODIPY-C11 was captured using a 525/50 and a 595/50 filter, respectively. At time 0, RSL3 (500 nM) or RSL3 (500 nM)+Ferrostatin (2 μM) were added and images were acquired every 30 seconds for a total of 30 minutes. Images were exported to ImageJ™ for analysis.

### Extracellular Flux Analysis and FAO Assay.

This process using a Seahorse™ XFe24 analyzer is described in depth elsewhere. A mitochondrial stress assay and fatty acid oxidation assay based of Agilent™ Technologies manual. Murine glioma cells (MG1 and MG3) were seeded in XFe24 cell culture microplates (Agilent™ Technologies) at 18,000 cells per well in 250 μL of BFP described above. After 4 hours, media was aspirated and replaced with either BFP or CMD BFP media. Treatments were continued for 18 hours. Mitochondrial stress tests were run with the following concentrations of media: 10 mM glucose, 2 mM glutamine, and 1 mM pyruvate in assay medium, and 2 μM oligomycin, 2 μM trifluoromethoxy carbonylcyanide phenylhydrazone (FCCP), and 0.5 μM rotenone/antimycin A. The assay involved injection of glucose (10 mM), followed by oligomycin (1 μM), followed by 50 mM 2-deoxy-d-glucose. Fatty acid oxidation assays were run using glucose (0.5 mM), glutamine (1 mM), 0.5 mM 1-carnitine and BSA conjugated palmitic acid.

### Animals, Orthotopic Tumor Implantation, and Diet Allocation.

Mice were anesthetized with ketamine/xylazine (100 mg/kg and 10 mg/kg, respectively) and assessed for lack of reflexes by toe pinch. Hair was shaved and scalp skin incised. The skull was cleaned with a Q-tip and bregma identified. A burr hole was made with a 17-gauge needle 2 mm lateral and 2 mm anterior to Bregma. Cell suspension was made from lifted adherent cell lines. Intracranial injection (5×10⁴ MG3 cells in 1 μL) performed under stereotactic guidance, 2 mm deep into the brain parenchyma aiming for subcortical white matter, using a Hamilton™ syringe at a flow rate of 0.25 μL/minute. Tumor growth was assessed through monitoring of luciferase signaling by bioluminescence imaging as previously described. Special diets were created by LabTest™ Diet (W.F. Fisher and Sons). A normal chow was used as a baseline. From this, two diets were created for experimental purposes. The diets used were a control diet with a defined 0.43% methionine and 0.33% cystine (w/w) and a cystine deprived-methionine restricted diet with 0.15% methionine and 0.0% cystine (w/w). Similar diets have shown safety in mouse experiments. Mice were transitioned to the diet seven days post tumor implantation. Investigators were not blinded to the allocation during experiments or outcome assessments.

### Tissue Collection.

Mice were assessed daily for signs of tumor morbidity. End-stage mice were anesthetized with intraperitoneal injection of ketamine/xylazine (100 mg/g and 10 mg/kg, respectively). Following cessation of toe pinch reflex, mice were perfused with PBS. The anterior-most portion of the brain, which encompassed the anterior most tip of tumor was sectioned and placed in 4% PFA. Remaining brains were harvested and placed on an aluminum weigh boat floating in liquid nitrogen for flash freezing.

### Targeted Metabolomic Profiling Sample Preparation In Vivo.

Tumor areas were cored out from whole frozen brains and weighed. Eighty percent HPLC grade methanol was added in 1.5 mL Eppendorf™ tubes with excised tumors. The tissue was homogenized and incubated at −80° C., then centrifuged at 14,000 rcf for 20 minutes at 4° C. Supernatant was transferred and a SpeedVac™ was used to remove excess liquid from the remaining metabolites.

### Sample Preparation In Vitro.

Two million MG1 or MG3 cells were plated on a 10 cm dish. Twenty-four hours after plating, cells were switched to control basal media or CMD basal media. Twenty-four hours after treatment, plates were washed twice with ice-cold PBS. Plates were aspirated, placed on dry ice and 1 mL of 100% HPLC grade methanol was added to the dish. Cells were scraped and transferred to cold Eppendorf™ tubes. Collection was done in matched pairs and the Eppendorf tubes were vortexed for 1 minute, placed on dry ice for 5 minutes, and vortexed again for 1 minute. Samples were spun at 14,000 ref for 20 minutes at 4° C. The supernatant was sent for LC-MS. Protein was extracted from pellets using cell extraction buffer with protease and phosphatase inhibitors. A colorimetric Bradford™ assay was read at 740 nm for evaluation of total protein content.

### LC-MS Data Acquisition and Processing.

Targeted LC-MS analyses were performed on a Q Exactive Orbitrap™ mass spectrometer (Thermo Scientific™) coupled to a Vanquish™ UPLC system (Thermo Scientific™). The Q Exactive operated in polarity-switching mode. A Sequan™ ZIC-HILIC column (2.1 mm i.d.×150 mm. Merck™) was used for separation of metabolites. The flow rate was set at 150 μL/minute. Buffers consisted of 100% acetonitrile for mobile B, and 0.1% NH₄OH/20 mM CH₃COONH₄ in water for mobile A. Gradient ran from 85% to 30% B in 20 minutes followed by a wash with 30% B and re-equilibration at 85% B. Data analysis was done using TraceFinder™ 4.1 (ThermoFisher™ Scientific). Metabolites were identified on the basis of exact mass within 5 ppm and matching the retention times with the standards. Relative metabolite quantitation was performed based on peak area for each metabolite. In vivo samples were normalized by weight and in vitro samples normalized by protein content using a Bradford™ assay. Data analysis was performed following log normalization and metabolite by metabolite mean subtraction. Metaboanalyst™ 5.0 (metaboanalyst.ca) was used for principal component analysis, differential assessment analysis, statistical tests, and quantitative pathway analysis.

### Global Quantitative Proteomics Analysis.

Tissue from mice with MG3 tumors placed on CMD or control diets was fixed at end stage in 4% PFA and paraffin-embedded. Five micromillimeter sections were made from blocks-tissue cores were scraped off slides and transferred to 1.5 mL Eppendorf™ tubes. Tissue lysis and de-crosslinking was performed according to known methods. Briefly, tissue was suspended in 50 μL of 5% SDS/300 mM Tris pH 8.5 and sonicated/boiled in a water bath (@ 90° C.×90 minutes). Samples were centrifuged then sonication/boiling was repeated (90° C.×10 minutes). The de-crosslinked lysate was centrifuged at 16.000×g in a benchtop centrifuge for 10 minutes and collected in a new Eppendorf™ tube. Cleared lysate was precipitated using the “salt method” as previously described. Pellets were resuspended in SDC lysis buffer (1% SDC. 10 mM TCEP, 40 mM CAA and 100 mM TrisHCl pH 8.5) and boiled for 10 minutes at 45° C., 1400 rpm to denature, reduce, and alkylate cysteine, followed by sonication in a water bath.

Samples were then cooled down to room temperature. Protein digestion proceeded overnight by adding LysC and trypsin in a 1:50 ratio (μg of enzyme to μg of protein) at 37° C. and 1400 rpm. Peptides were acidified by adding 1% TFA and vortexing followed by StageTip™ clean-up via SDB-RPS. Peptides were loaded on one 14-gauge StageTip™ plugs. Peptides were washed two times with 200 μL 1% TFA 99% ethyl acetate followed by 200 μL 0.2% TFA/5% ACN in centrifuge at 3000 rpm, followed by elution with 60 μL of 1% Ammonia, 50% ACN into Eppendorf™ tubes and dried at 60° C. in a SpeedVac™ centrifuge. Peptides were resuspended in 7 μL of 3% acetonitrile/0.1% formic acid and injected on Thermo Scientific™ Orbitrap Fusion™ Tribrid™ mass spectrometer using the DIA method for peptide MS/MS analysis. The UltiMate™ 3000 UHPLC system (ThermoFisher™ Scientific) and EASY-Spray™ PepMap RSLC C18 50 cm×75 μm ID column (ThermoFisher™ Scientific) coupled with Orbitrap™ Fusion were used to separate fractionated peptides with a 5-30% acetonitrile gradient in 0.1% formic acid over 120 minutes at a flow rate of 250 nL/min. After each gradient, the column was washed with 90% buffer B for 5 minutes and re-equilibrated with 98% buffer A (0.1% formic acid, 100% HPLC-grade water) for 40 minutes. Survey scans of peptide precursors were performed from 350-1200 m/z at 120K FWHM resolution (at 200 m/z) with a 1×10⁶ ion count target and a maximum injection time of 60 ms. The instrument was set to run in top speed mode with 3s cycles for the survey and the MS/MS scans. After a survey scan, 26 m/z DIA segments were acquired from 200-2000 m/z at 60K FWHM resolution (at 200 m/z) with a 1×10⁶ ion count target and a maximum injection time of 118 ms. HCD fragmentation was applied with 27% collision energy and resulting fragments were detected using the rapid scan rate in the Orbitrap™. The spectra were recorded in profile mode. DIA data were analyzed with direct DIA 2.0 (Deep learning augmented spectrum-centric DIA analysis) in Spectronaut™ Pulsar X, a mass spectrometer vendor independent software from Biognosys™. The default settings were used for targeted analysis of DIA data in Spectronaut™ except the decoy generation was set to mutated. The false discovery rate (FDR) will be estimated with the mProphet™ approach and set to 1% at peptide precursor level and at 1% at protein level.

Results obtained from Spectronaut™ were further analyzed using the Spectronaut™ statistical package. Significantly changed protein abundance was determined by unpaired t-test with a threshold for significance of p<0.20 (permutation-based FDR correction) and 0.58 log 2FC.

### Desorption Electrospray Ionization-Imaging Mass Spectrometry (DESI-IMS) Tissue Preparation.

Consecutive coronal brain sections were cut at −20° C. into 12 μm thick sections on a cryostat (Leica™), and directly thaw-mounted onto SuperFrost Plus™ glass Microscope Slides (Fisherbrand™). Before analysis, the sections were dried under vacuum in a desiccator for 15 minutes. High resolution mass spectrometry with desorption electrospray ionization (DESI) source was used to scan slices. After DESI-MSI, the tissue sections were stained with Hematoxylin and Eosin (H&E). A clinical pathologist (PC) identified and outlined tumor regions. The identified region was superimposed upon DESI-MSI maps to extract specific quantitative morphometry allowing for statistical comparisons between tumor regions of CMD mice versus control mice.

### DESI-IMS Data Acquisition and Processing.

The tissue sections were imaged at 50 μm resolution on a Prosolia™ 2D-DESI source mounted on the SYNAPT G2-Si q-ToF ion mobility mass spectrometer. The electrospray solvent consisted of methanol/water/formic acid (98:2:0.01; v/v/v) containing 40 pg/pL of leucine enkephalin as internal lock mass. The flow rate was 2 μL/minutes. The spray capillary voltage was set to 0.6 kV, the cone voltage was 50 V, and the ion source temperature was set to 150° C. Mass spectra were acquired using negative ionization mode with the mass range of m/z 50 to 1200. DESI imaging of all tissue samples were run in a randomized order using the same experimental conditions in duplicates. Ion image mass spectral data (corresponding m/z features in every pixel within the image) from DESI-MSI was processed for visualization using Waters™ High Definition Imaging (HDImaging™, V1.5) software. The images were normalized to the total ion current. Group differences were calculated using a two-tailed parametric Welch's t-test with a false discovery rate (FDR) of 0.05 or less as significant. The lipid ions were annotated by searching monoisotopic masses against the available online databases such as METLIN and Lipid MAPS with a mass tolerance of 5 ppm and also matching the drift times with the available standards.

### Example 2. CMD Sensitizes Glioma Cells to Ferroptosis Induction

The effects of CMD on glioma responsiveness to ferroptosis were examined. Given that cysteine and methionine are necessary for the synthesis of glutathione, the substrate used by the enzyme GPX4 for detoxification of lipid peroxides, CMD should synergize with GPX4-mediated ferroptosis induction. To test this, media was adapted for cell culture based on the previous ferroptosis permissive glioma culture methods. The responsiveness of human and murine glioma cell lines to ferroptosis induction was surveyed in the presence and absence of cysteine/methionine. See Table 4, below.

TABLE 4
Cell Line Designations Used.

| Designation | Nomenclature | Species | Genetic Background | Details |
|---|---|---|---|---|
| 333 | Mouse- | Mouse | P53−/−, PDGFA | Diffusely infiltrating |
| [36] | glioma-1 | | overexpressing | phenotype |
| | (MG1) | | | |
| ACre | MG2 | Mouse | P53−/−, PDGFA | Diffusely infiltrating |
| | | | overexpressing | phenotype |
| APCL | MG3 | Mouse | P53−/−, PDGFA | Diffusely infiltrating |
| | | | overexpressing | phenotype |
| MGPP3 | MG4 | Mouse | P53−/−, PTEN−/−, | Aggressive, |
| [35] | | | PDGFB overexpressing | pseudopalisading |
| | | | | necrosis |
| TS543 | TS543 | Human | Human GBM culture; | Proneural |
| | | | PDGFR-A amplified | |
| KNS42 | KNS42 | Human | Pediatric GBM culture; | Mesenchymal |
| | | | p53 mutated; H3 | |

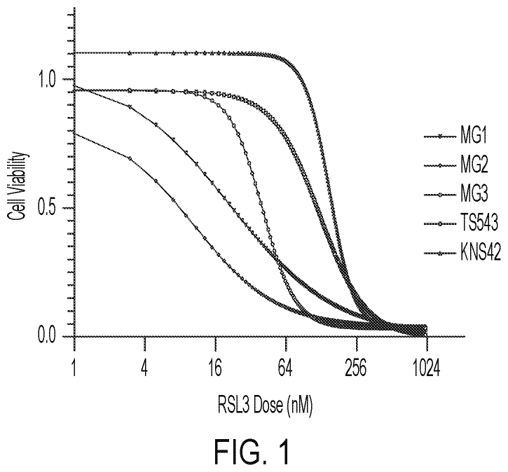

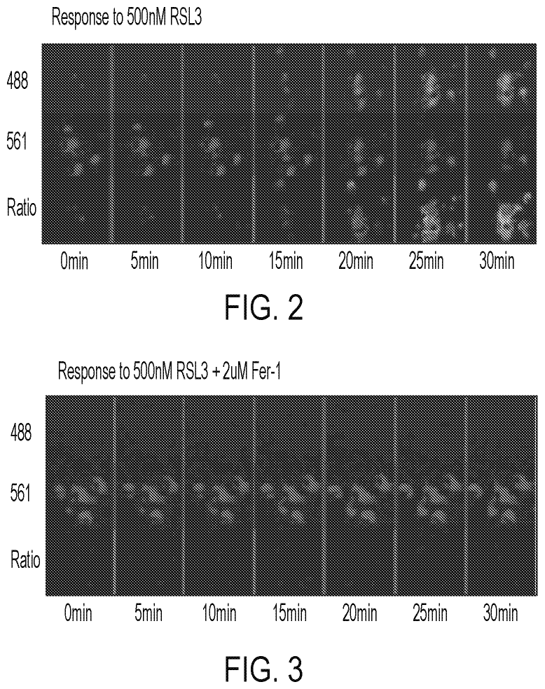

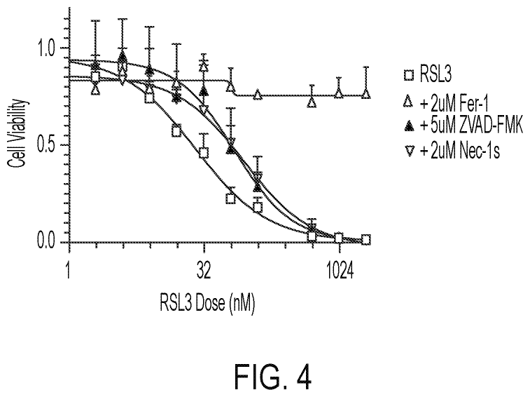

Five of five cell lines assayed had baseline sensitivity to ferroptosis by the GPX4 inhibitor RSL3. See FIG. 1, which shows the results of 384-well close-response curves showing response to RSL3 from 6 glioma cell lines: MG11, MG2, MG3, TS543, and KNS42. This was confirmed by live-cell confocal microscopy showing RSL3 mediated induction of lipid peroxidation as evidenced by green fluorescence shift in the Bodipy-Ci 1 dye following addition of RSL3. See FIG. 2, showing live cell confocal microscopy of Bodipy-Ci 11 labeled MG1 cells treated with 500 nM RSL3, added at time 0 minutes. Ferrostatin, a ferroptosis inhibitor, prevented this lipid peroxidation. See FIG. 3, which shows live cell confocal microscopy of Bodipy-C11 labeled MG1 cells with 500 nM RSL3 and 2 uM Ferrostatin-1 added at time 0 minutes. The upper panels show the oxidized, middle panels the reduced, and bottom panels the ratio of oxidized/reduced Bodipy-C11. Each frame=100 μm-100 μm. RSL3 mediated cell death, however, was not rescuable by necroptosis inhibitors (Nec-1s) or apoptosis inhibitors (ZVAD-FMK). See FIG. 4 which provides data on the representative close-response of MG1 cells treated with RSL3 (red), RSL3 plus Ferrostatin-1 (blue), RSL3 plus 5 uM ZVAD-FMK (black), RSL3 plus 2 uM Nec-1s (gray).

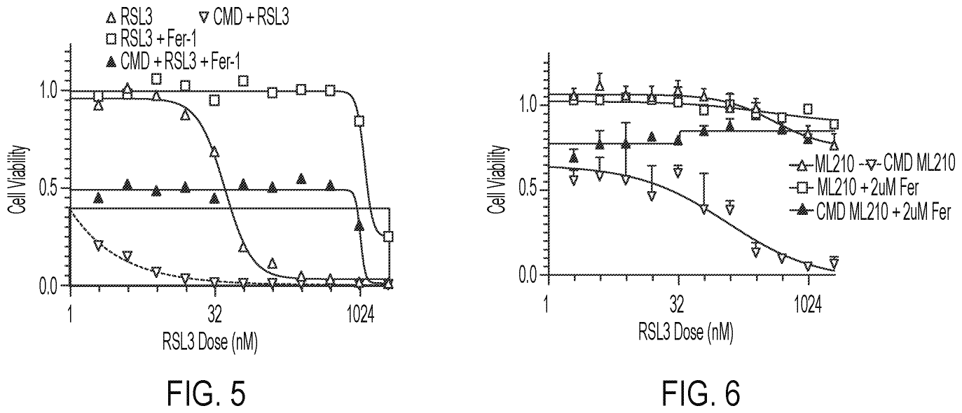

Dose response assays demonstrated that RSL3 and ML-210, another GPX4 inhibitor, both had synergistic enhancement of ferroptosis with CMD (FIG. 5 and FIG. 6). FIG. 5 shows representative 384-well close-response showing MG3 cells treated with RSL3 (red), RSL3 plus 2 uM Ferrostatin-1 (brown), CMD plus RSL3 (blue), CMD plus RSL3 and 2 uM Ferrostatin-1 (orange). FIG. 6 is a representative close-response curve showing MG3 cell responses to ML-210 (red), ML-210 plus 2 uM Ferrostatin-1 (brown), CMD+ML-210 (blue), CMD+ML-210+2 uM Ferrostatin-1 (orange).

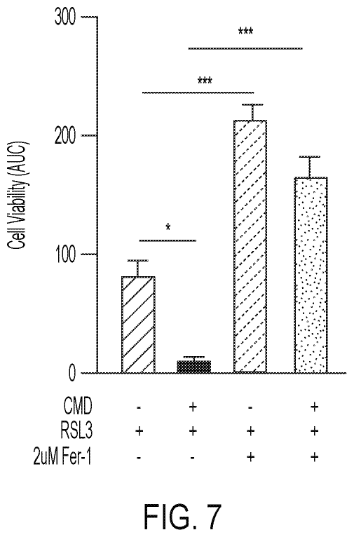

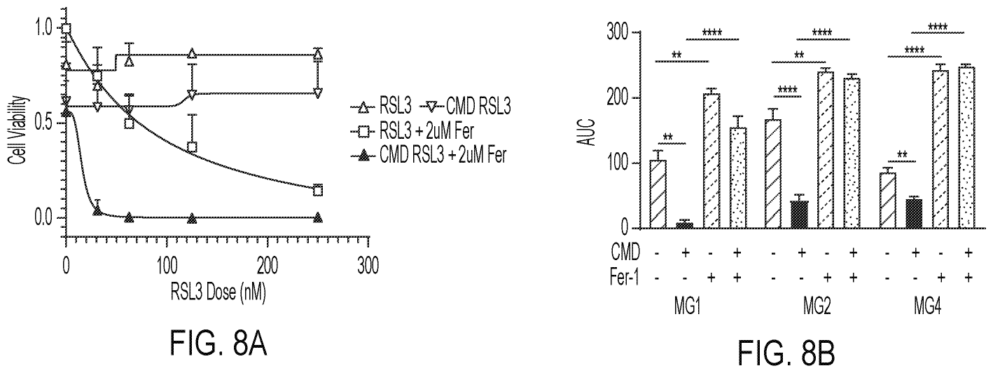

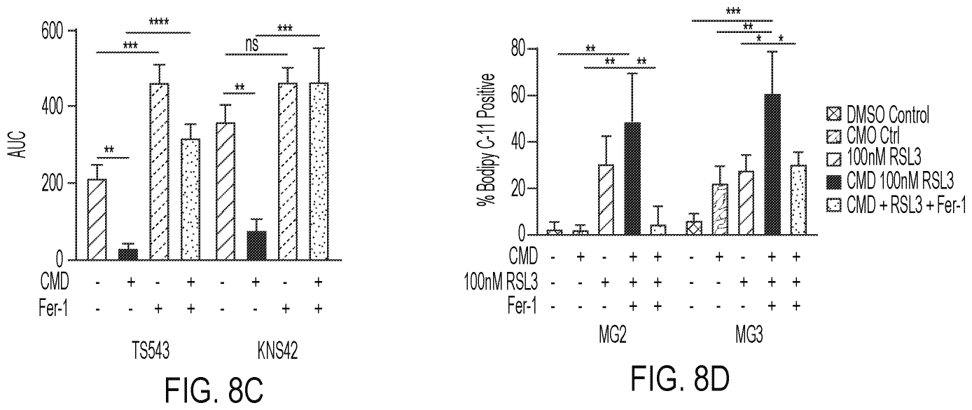

Increased sensitivity to RSL3 mediated ferroptosis by CMD was seen in all responsive murine and human glioma cell lines (see FIG. 7 and FIG. 8A, FIG. 8B, FIG. 8C). FIG. 7 presents AUC quantification for close response curves from 3-independent 96-well close response curves of MG3 murine glioma cell lines treated with RSL3±CMD±2 μM Ferrostatin-1. FIG. 8A shows representative close-response curves for MG1 glioma cells treated with RSL3±CMD±2 uM Ferrostatin-1. FIG. 8B shows the AUC quantification for close-response curves from three murine glioma cell lines treated with RSL3±CMD±2 μM Ferrostatin-1. FIG. 8C shows AUC quantification for close response curves three human glioma cell lines treated with RSL3±CMD±2 μM Ferrostatin-1. FIG. 8D is a quantitation of 3 independent flow cytometry experiments using Bodipy-C11 for two additional murine glioma cell lines (MG2, MG3).

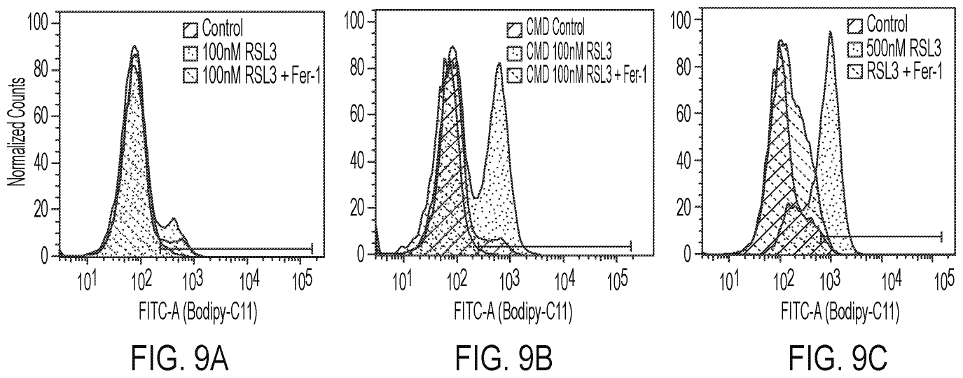

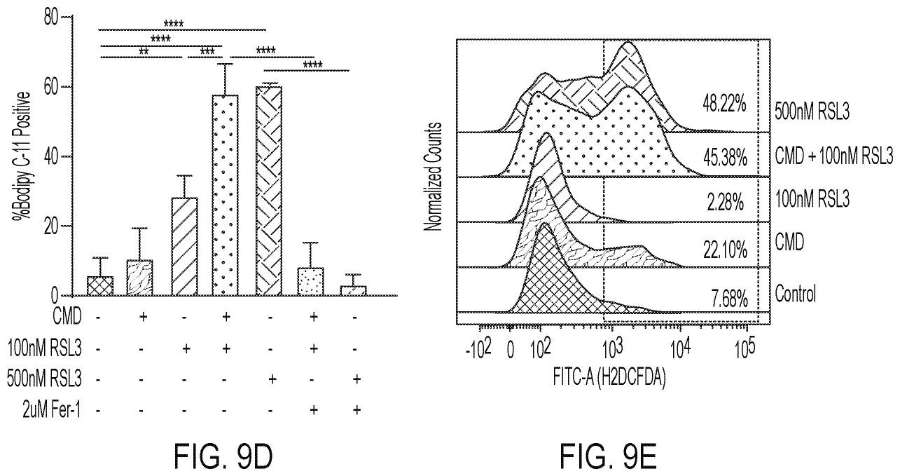

Pre-treatment incubation of glioma cells for 6 hours in CMD sensitized tumor cells to subthreshold doses of RSL3 across all murine glioma cell lines as determined by Bodipy-C11 fluorescence shift (FIG. 9A and FIG. 9B; FIG. 8D). FIG. 9A presents representative Bodipy-C11 flow data from MG1 cells: left panel shows DMSO control (red), 100 nM RSL3 (blue), and 100 nM RSL3 plus 2 uM Ferrostatin-1 (orange) treatment for 30 minutes. The middle panel shows the same conditions but with 6 hours of cysteine methionine deprivation pretreatment. Right panel shows a higher dose of RSL3 treatment (500 nM). FIG. 9B is the quantitation of 3 independent experiments demonstrated in FIG. 9A.

Next, an ex vivo organotypic slice culture model from a human primary glioblastoma was use to further validate the effects of CMD. The slices were treated with RSL3 and assayed via flow cytometry for levels of reactive oxygen species (ROS) using H2DCFDA. Similar to the in vitro results, a low dose of RSL3 (100 nM) plus CMD increased ROS to levels equivalent to a high dose of RSL3 (500 nM). See FIG. 9C, which presents flow cytometry results for tests using H2DCFDA of ex vivo organotypic slice cultures from a human primary glioblastoma (CUMC TumorBank 6193) cultured in control or CMD media and treated with RSL3. In the primary ex vivo samples, CMD alone was sufficient to increase ROS levels.

### Example 3. CMD Induces Transcriptional Changes Canonically Associated with Ferroptosis

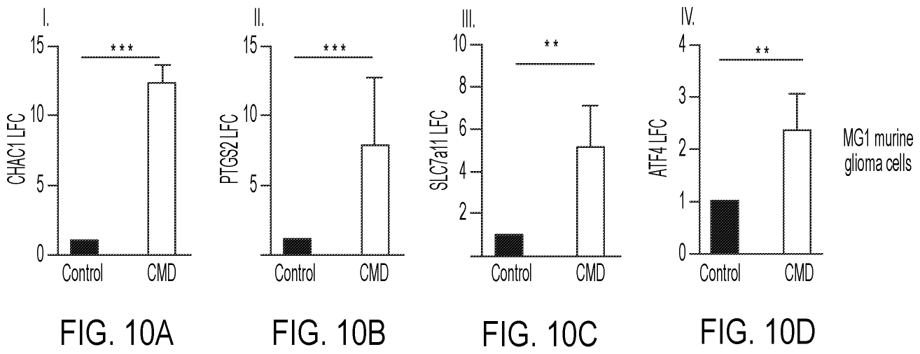

The transcriptional hallmarks of cellular response to CMD were investigated. Previous studies have shown that CHAC1, PTGS2, and SLC7a11 mRNAs are upregulated following ferroptotic induction. Furthermore, ATF4 has been tied to amino acid deprivation and ferroptotic stress response as a mechanism to increase SLC7a11 expression and cysteine import. mRNA was harvested following 24 hours of CMD in the murine glioma cells and 48 hours of CMD in the human glioma cells. RT-qPCR of the murine glioma cells showed that by 24 hours there were significant increases in CHAC1, PTGS2, SLC7a11 and ATF4 transcripts. See FIG. 10, which presents RT-qPCR data for (A) CHAC1, (B) PTGS2, (C) SLC7a11, and (D) ATF4 transcripts from MG1 cells in either control (black) or 24 hour CMD (grey) conditions.

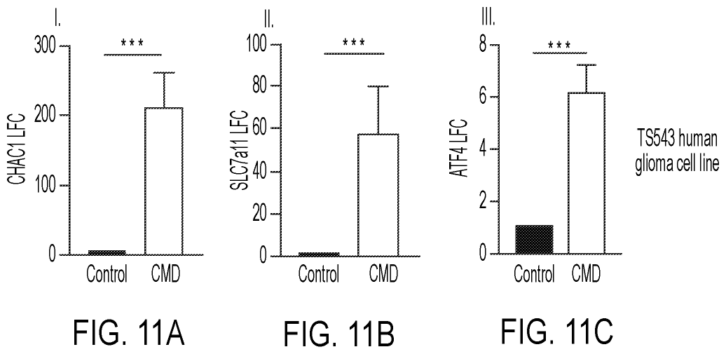

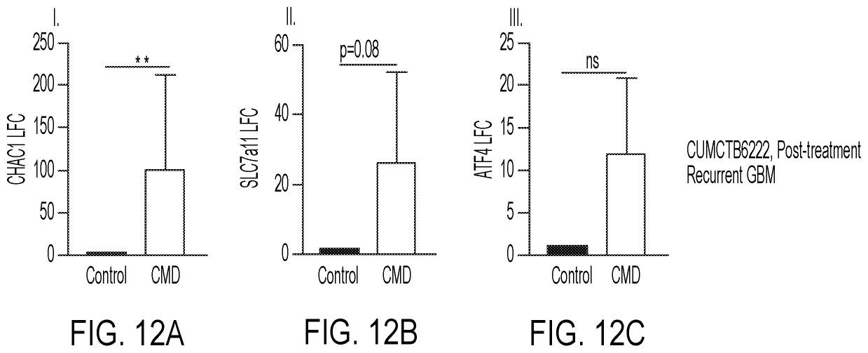

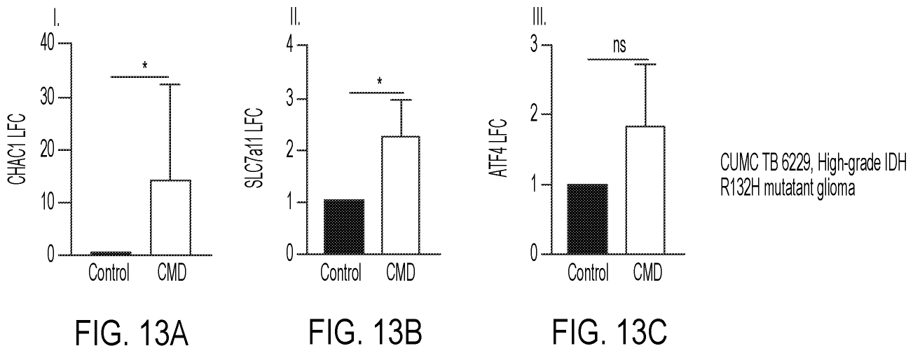

In the human glioma cells a significant upregulation of CHAC1, SLC7a11 and ATF4 transcripts were seen at 48 hours (FIG. 11; RT-qPCR data for TS543 cells after 48 hours CMD (grey) compared to control (black) for (A) CHAC1, (B) SLC7A11, and (C) ATF4 transcripts). These changes were also seen in the ex vivo setting, where organotypic slices were generated from a post-treatment recurrent GBM (FIG. 12) and a high-grade R132H IDH1 mutated glioma (FIG. 13) with neighboring slices being placed into either control media or CMD media. After 24 hours, RNA was harvested and RT-qPCR showed significant increases in CHAC1 (see FIG. 12; RT-qPCR data of ex vivo organotypic slices for CUMC Tumor Bank 6229 Post-treatment recurrent glioblastoma treated in control (black) or CMD (gray) media. Transcripts for (A) CHAC1, (B SLC7a11, and (C) SLC7a11 shown) and FIG. 13A). The IDH1-mutated glioma had significantly increased SLC7a11 expression following CMD, while the IDH1-wild-type glioma trended towards an increase of SLC7a11 (p=0.08) (FIG. 12 and FIG. 13B). FIG. 13 shows RT-qPCR data of ex vivo organotypic slices for high-grade R132H mutant glioma, CUMC Tumor Bank 6234 ex-vivo organotypic slices in control or CMD media. Transcripts for (A) CHAC1, (B) SLC7a11, and (C) ATF4 shown. Data for FIGS. 10, 11, 12, and 13 are plotted as mean of log fold change ±SEM, n=3 independent experiments for A-B and three independent slices for C,D. Statistics assessed using t-test on the un-transformed dCT values. These findings show that CMD induces transcriptional hallmarks of ferroptosis and an integrated stress response in murine and human gliomas in in vitro and ex vivo settings.

### Example 4. CMD Alters Glioma Cell Metabolism

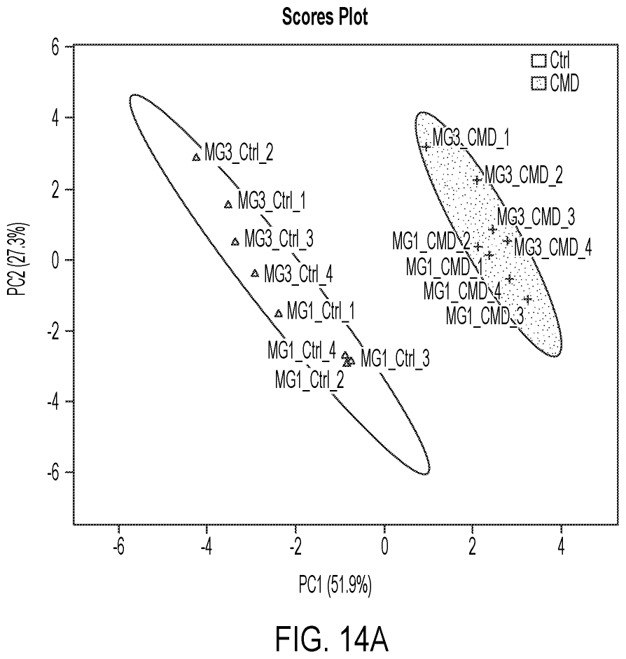

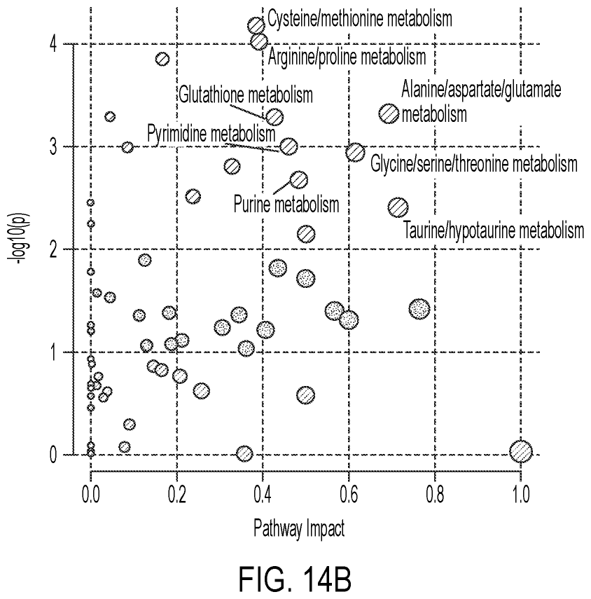

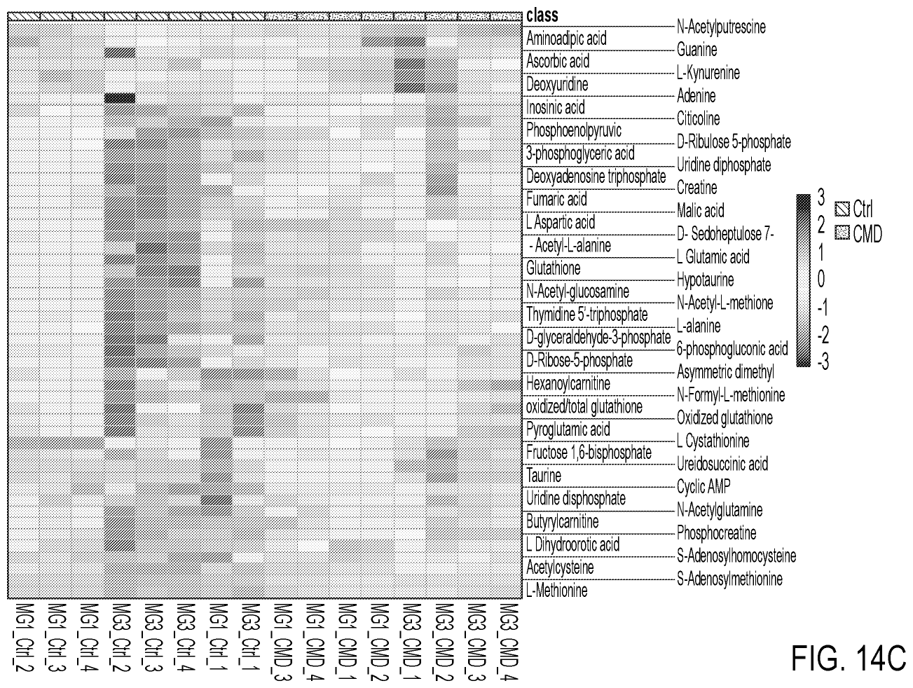

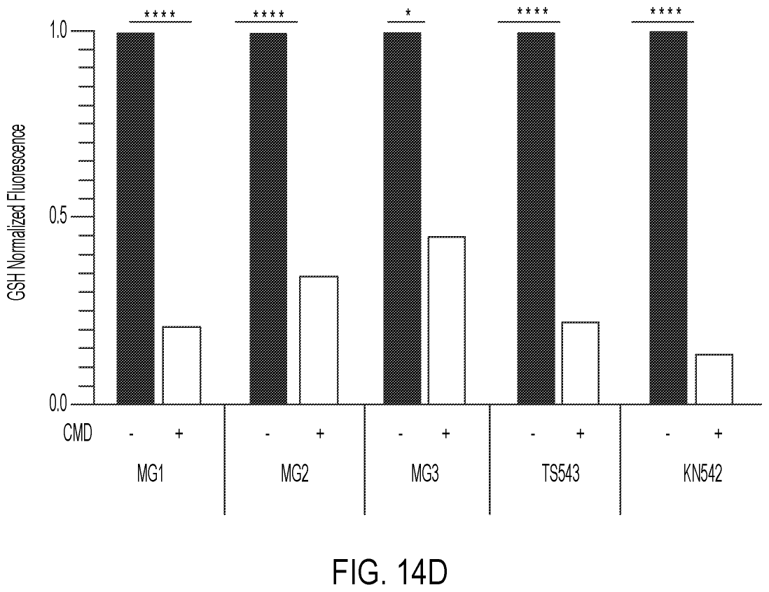

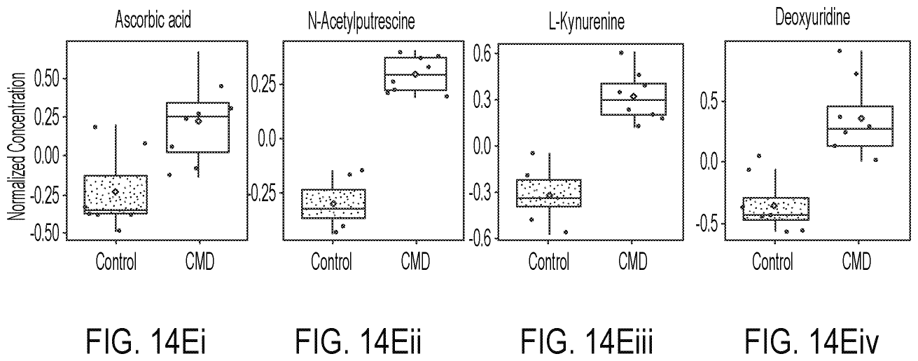

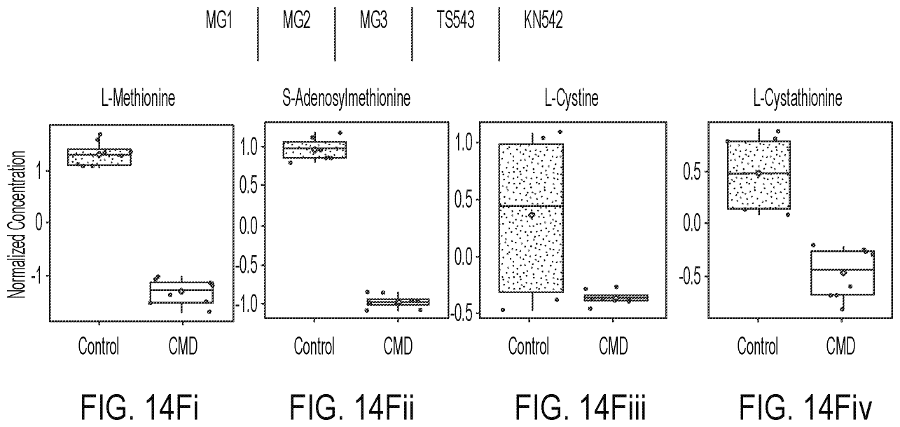

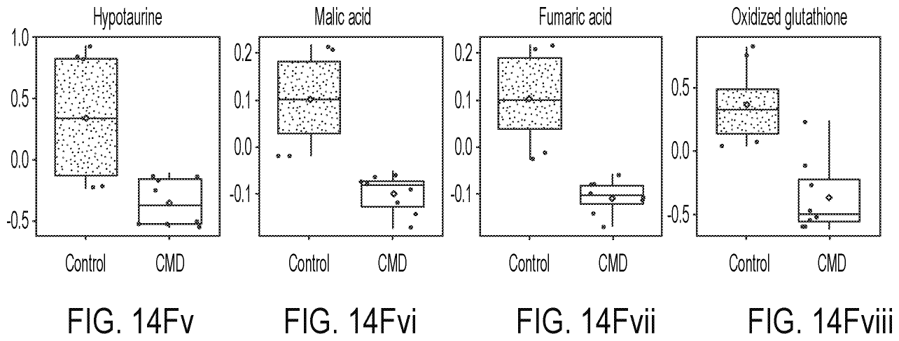

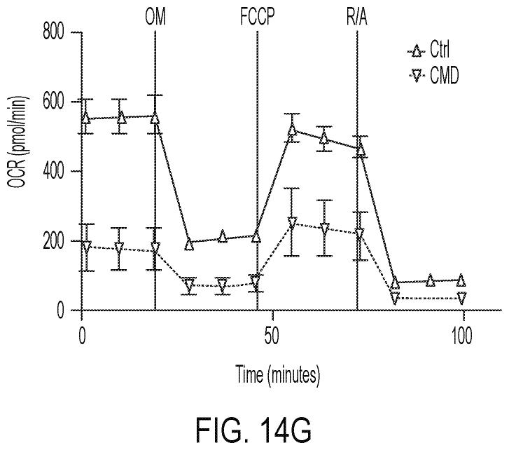

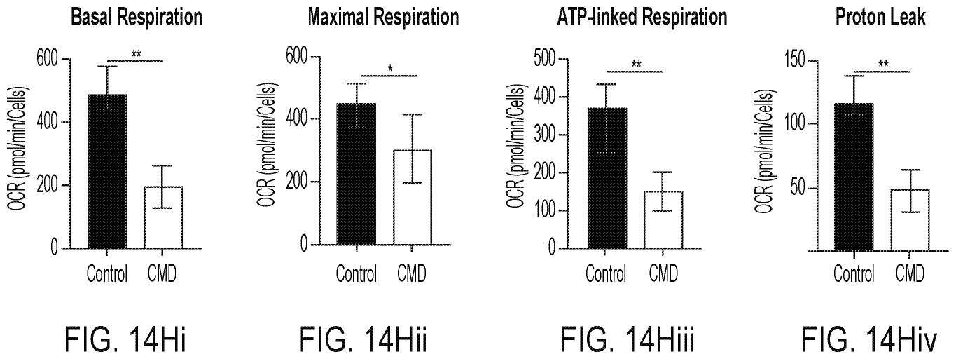

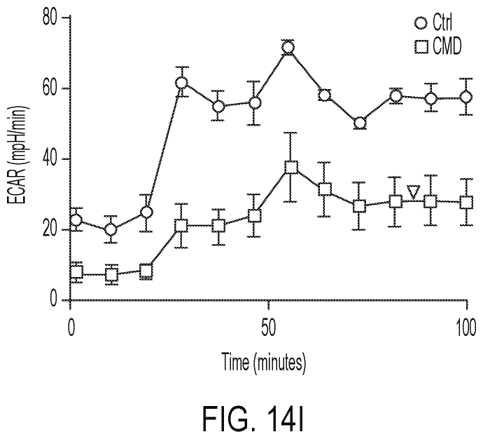

To characterize further the effects of CMD on glioma cells, targeted metabolite profiling was performed on two murine cell lines (MG1, MG3) treated with CMD for 24 hours. Principal component analysis of treated and untreated samples demonstrated clear clustering of metabolites according to treatment condition. See FIG. 14A, a principal component analysis of targeted metabolite profiling showing clustering along treatment conditions (light grey=control, dark grey=CMD).

An enrichment ratio based upon the number of differentially assessed metabolites within specific metabolite pathways showed that cysteine/methionine metabolism, glycine-serine-proline metabolism, taurine/hypotaurine metabolism, alanine/aspartate/glutamate metabolism and seleno-compound metabolism were significantly impacted by CMD (FDR-corrected p-value <0.05). See FIG. 14B, a pathway analysis of targeted metabolite profiling across control and CMD samples spanning 200 metabolites with relative concentrations log transformed and samples scaled by mean. Labeled pathways have FDR<0.05.

The heatmap of the top 50 differentially assessed metabolites showed clear separation between CMD and control samples. As expected, glutathione (oxidized and reduced) was significantly reduced by CMD (LFC 0.124; FDR-corrected p-value <0.05). See FIG. 14C. FIG. A3C-14C (a heatmap showing top 50 differentially assessed metabolites based on FDR-corrected p-value, all <0.05.) and FIG. 14D (a calorimetric assay of reduced glutathione levels for (left to right) MG1, MG2, MG3, TS543, and KNS42 in control (black bars) and CMD treated cells after 24 hours (gray bars)) show that the top upregulated metabolites (ascorbic acid, n-acetylputrescine, 1-kynurenine, deoxyuridine; FIG. 14E), were closely tied to the citric acid cycle. See FIG. 14E, showing the normalized metabolite concentrations for key metabolites upregulated in CMD versus control, all with FDR<0.05.

The top downregulated metabolites (methionine, s-adenosyl methionine, 1-cystine, l-cystathionine, hypotaurine, oxidized glutathione; FIG. 14F) were closely tied to the glutathione synthesis, cysteine/methionine metabolism including the trans-sulfuration pathway. See FIG. 14F, showing the normalized metabolite concentrations for key metabolites downregulated in CMD versus control, all with FDR<0.05.

These findings show that CMD can not only impact a host of metabolic pathways but also impacts cellular energetic metabolism. Previous studies have shown that acute oxidative stress can oxidize cysteine residues on proteins necessary for the electron transport chain and citric acid cycle. Thus, CMD should dampen cellular metabolism. Using a mitochondrial stress assay with the Seahorse™ Analyzer on the murine glioma cells, we measured basal oxygen consumption followed by sequential measurements of ATP-production (oligomycin inhibition), maximal respiration (FCCP inhibition) and mitochondrial respiration (rotenone/antimycin inhibition) (FIG. 14G). Basal respiration, maximal respiration, ATP-linked respiration and proton-leak were all significantly reduced with CMD (FIG. 14H). Importantly, the extracellular acidification rate also decreased, showing a dampening of both aerobic and anaerobic respiration, supporting a global effect of CMD on glioma cell metabolism. See FIG. 14I. FIG. 14G, FIG. 14H, and FIG. 14I show a Seahorse Mitochondrial stress test of MG3 cells in either control (black) or 12 hours CMD (gray) OM: oligomycin, FCCP: Carbonyl cyanide-4 (trifluoromethoxy) phenylhydrazone, R/A: rotenone and antimycin A (n=5). FIG. 14H shows the basal respiration, maximal respiration, ATP-linked respiration and proton leak values calculated from the experiment in FIG. 14I were calculated and normalized (n=5 per group). In FIG. 14I, the extracellular acidification rate for control (black) or 12 hour CMD (gray) is shown.

### Example 5. CMD Leads to Increased Survival In Vivo

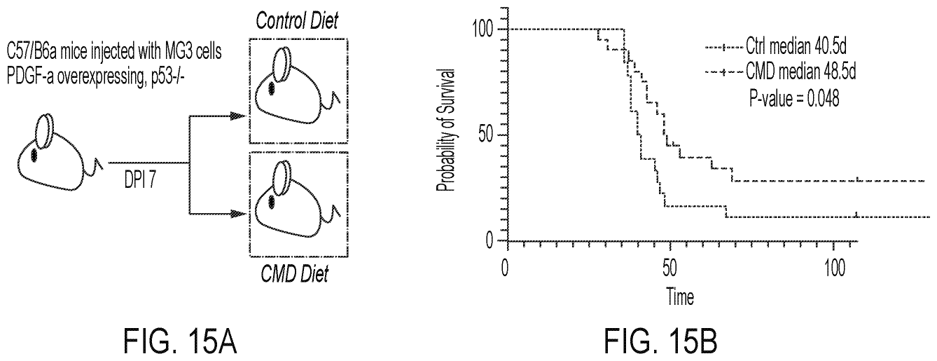

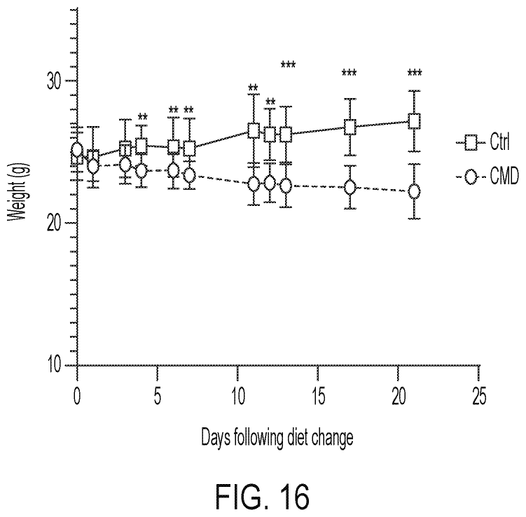

The effects of dietary CMD on survival were tested. MG3 cells were orthotopically injected into mice. At 7 days post injection (DPI), the mice were either switched to a control diet (0.43% w/w methionine, 0.40% w/w cystine) or a CMD diet (0.15% w/w methionine, 0% w/w cystine). See FIG. 15A, a diagram of the experimental paradigm. The diet was tolerated with no adverse effects, though notably CMD mice maintained lower weights than control mice (see FIG. 16, which shows the weights from C57/B6 male mice put on control or CMD diet. T-tests performed to assess significance. **p<0.01, *** p<0.001).

Kaplan-Meier survival analysis showed a significant survival benefit for CMD mice over control mice, despite the lower weights of the CMD mice (Median Survival: control-40 DPI, CMD-8 DPI; p=0.048). See FIG. 15B a Kaplan-Meier curve outlining survival comparing control (n=18; 8 male, 10 female; red) versus CMD (n=20; 10 male, 10 female; blue) diet mice orthotopically injected with MG3 cells (Median Survival: Control—40 days, CMD—48 days; p=0.048).

### Example 6. CMD Induces Changes within Tumor Lipidome, Metabolome and Proteome

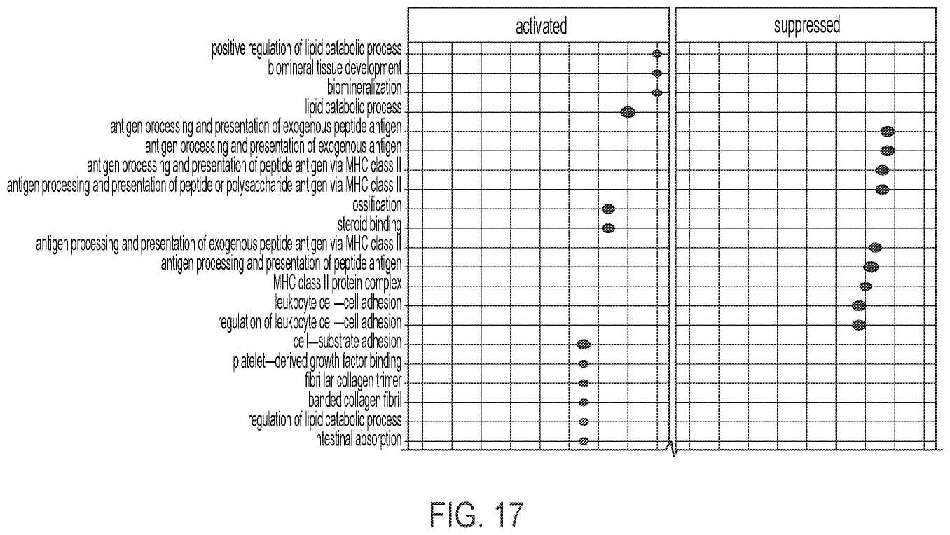

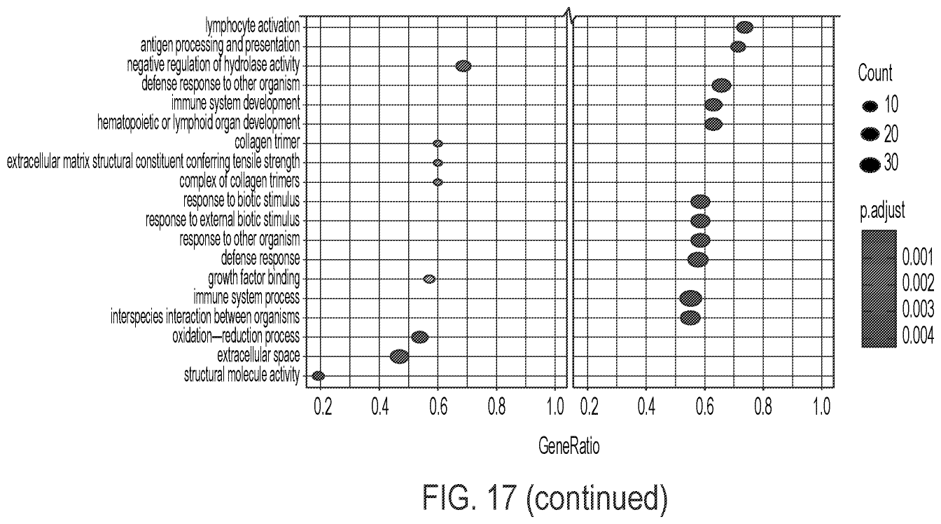

To explore changes induced by CMD on glioma cell phenotype at the molecular level, proteomic analysis of adjacent sections from control (n=3) and CMD (n=4) mice was performed. All tissue based analyses were performed on male mice to control for potential sex-specific metabolic effects. CMD induced alterations in numerous protein species (299 protein species differentially expressed; FDR-corrected p-value 0.20, ILFC|>0.58). Of the pathways activated in CMD versus control, the one with the greatest enrichment score was lipid catabolic processes. See FIG. 17. Other gene/protein sets enriched in CMD versus control included oxidation/reduction processes, positive regulation of lipid catabolism and cell substrate adhesion and extracellular space. See FIG. 17. CMD led to a robust immunosuppressive signature involving downregulation of proteins related to antigen presentation and lymphocyte activation (FIG. 17).

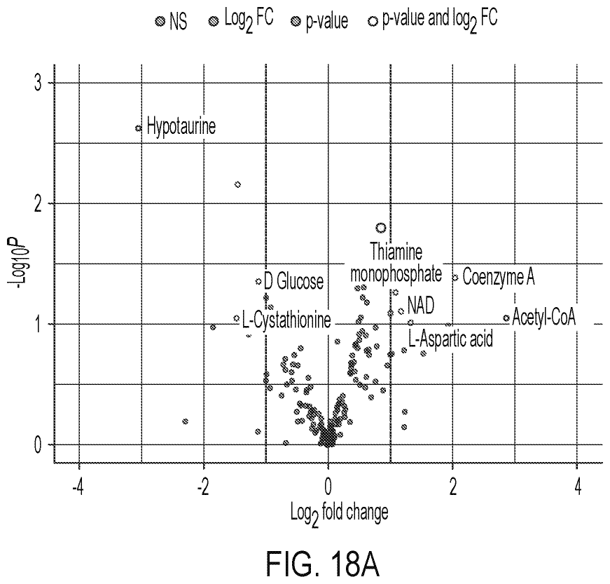

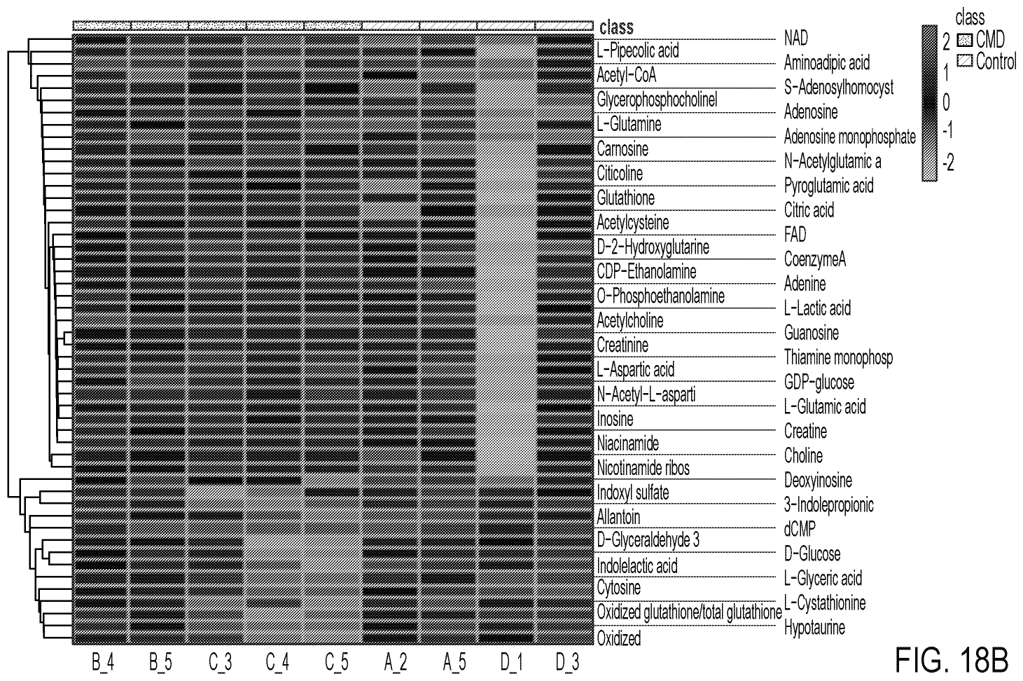

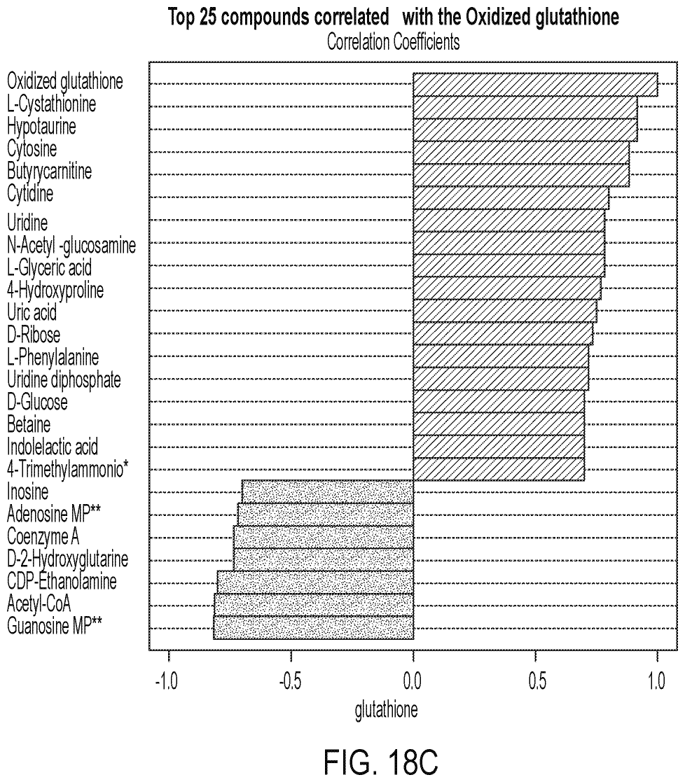

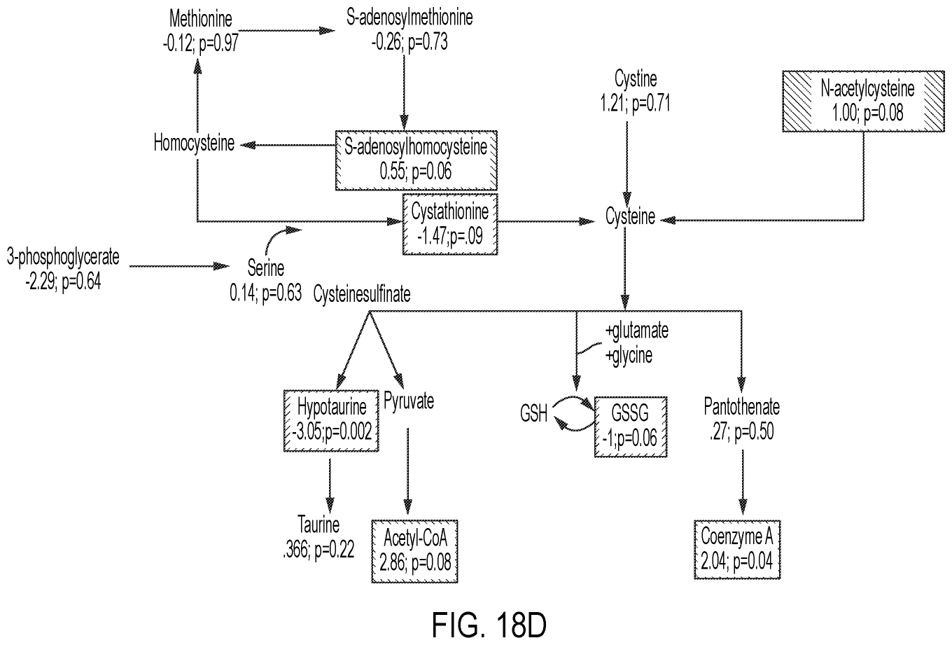

Targeted metabolite profiling on flash frozen tumor tissue harvested from end-stage MG3 tumor bearing mice (control n=4 mice, CMD n=5 mice). A volcano plot (FIG. 18A; labeled metabolites having p<0.1 and log fold change (LFC)>1) and a heatmap (FIG. 18B, showing the top 50 differentially assessed metabolites) demonstrate the top differentially assessed metabolites. L-cystathionine and hypotaurine were positively correlated with oxidized glutathione levels, while acetyl CoA and coenzyme A were strongly negatively correlated with oxidized glutathione. See FIG. 18C, which shows the correlation between oxidized glutathione and associated metabolites altered.

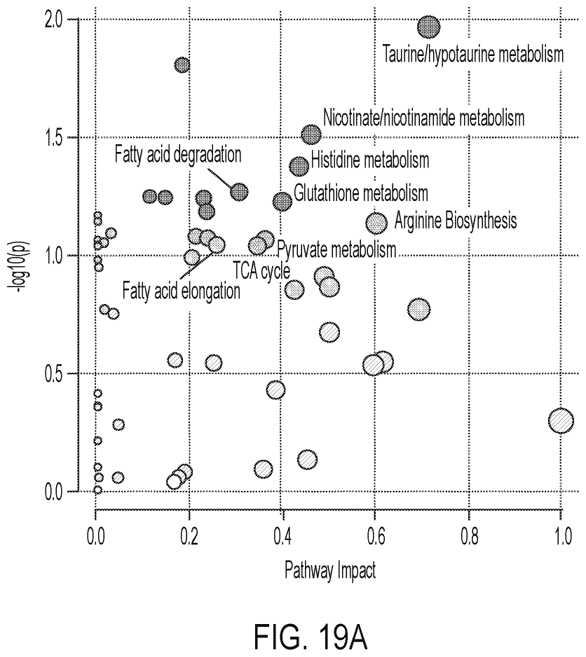

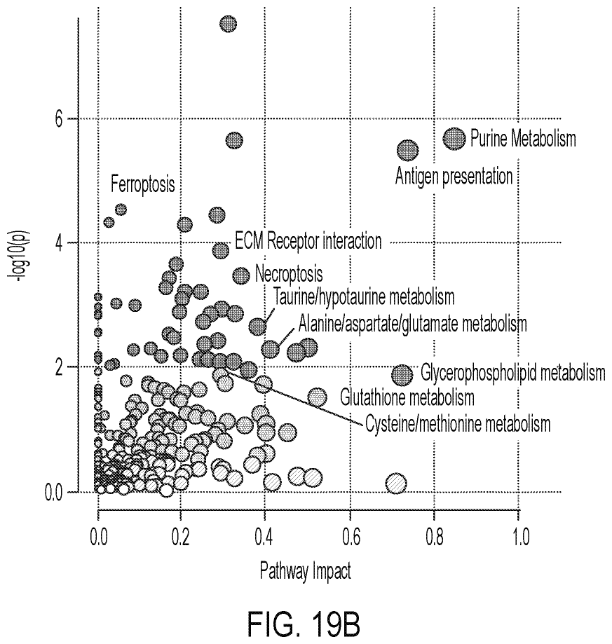

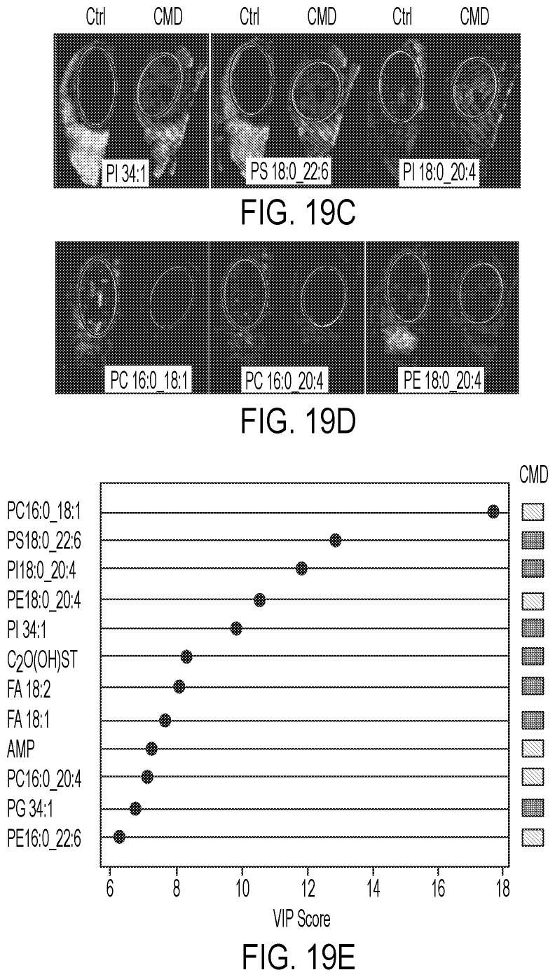

FIG. 18D is a schematic of cysteine metabolism with key differentially assessed metabolites with Log2FC and t-test p-value listed between control (n=4) and CMD (n=5). Quantitative metabolite pathway analysis showed key changes within taurine/hypotaurine metabolism, glutathione metabolism, arginine metabolism, the TCA cycle, and fatty acid elongation/degradation (p<0.10) (see FIG. 19A, a pathway analysis of targeted metabolite profiling across control (n=4) and CMD (n=5) male mice spanning 200 metabolites with relative concentrations log transformed and samples scaled by mean. Pathways enriched with p<0.05 are labeled. A joint pathway analysis combining the proteomic differential expression matrix with FDR<0.2 and ILFC|>0.58 and the metabolomic differential expression matrix of compounds with ILFC>0.58| yielded a comprehensive tissue level view of pathways altered by the diet. See FIG. 19B, showing a joint pathway analysis combining proteomics data of differential expression analysis comparing CMD vs. control (FDR<0.2, |LFC|>0.58) and metabolite differential assessment analysis (|LFC|>0.58) comparing CMD vs. control. Enrichment analysis using hypergeometric test and integration method based on queries. Relevant pathways with FDR<0.1 labeled.

Cysteine/methionine metabolism, glutathione metabolism, ferroptosis, glycerophospholipid metabolism were relevant pathways significantly altered based on the joint proteomic and metabolomic analysis. Thus, on both a metabolite and protein level, CMD led to profound alteration of the tumor microenvironment.

To identify spatial distributions of metabolites and lipids in the tumor tissues and to further analyze metabolic changes induced by ferroptosis, we performed mass spectrometry imaging experiment. Desorption electrospray ionization imaging mass spectrometry (DESI-IMS) was carried out on six end-stage MG3 tumor samples (Control N=3, CMD N=3) in both positive and negative ion modes. Overall, the major detected ions in mouse glioma tissues were lipids including saturated and unsaturated free fatty acids (FFA), phosphatidylcholines (PC), phosphatidylethanolamines (PE), and phosphatidylinositols (PI), phosphatidylserines (PS), phosphatidylglycerols (PG) and sulfatides (S). The results outlined are from the negative ion mode where more significant changes in lipid abundances were observed between control and CMD groups. The abundance distribution maps for a CMD and a control slice with tumor areas outlined are shown for lipid species increased in CMD (FIG. 19C, showing representative DESI-MS images from tumor region overlay included for upregulated lipid species) and lipid species decreased in CMD (FIG. 19D, showing representative DESI-MS images from tumor region overlaid included for downregulated lipid species). Variable importance of projection plots for significantly altered lipid species are shown in FIG. 19E. The variable importance of projection shows lipid species important in discriminating the two classes of samples apart (FDR-corrected p-value <0.05) from 6 male mice (control n=3, CMD n=3) with data from negative ion mode shown.

Among the identified lipid species, the relative abundance of several lipid species including FA 18:2, FA 18:1, PS 18:22:6, PI 18:0_20:4, PI 34:1, PG 34:1, and C20(OH)ST were significantly increased in the tumor regions of the CMD group compared to the control group. In contrast, the relative abundances of PC 16:0_18:1, PE 18:0_20:4, PC 16:0_20:4, and PE 16:0_22:6, and adenosine monophosphate (AMP) were significantly decreased (FDR-corrected p-value <0.05) in the tumor regions of CMD mice compared to the tumors of control mice. See FIG. 19C through FIG. 19E.

### Example 7. CMD Leads to Lower Levels of Glutathione (GSH)

A basis for the invention that depriving glioma cells of sulfur containing amino acids (cystine and methionine) will result in lower levels of glutathione (which normally protects cells from lipid peroxidation) and therefore would sensitize the glioma cells to ferroptosis inducing drugs. To demonstrate this, cells were stained with Bodipy C-11, a fluorescent probe for membrane-localized ROS, which was used as a marker of ferroptosis, and performed flow cytometry. The results showed that CMD sensitized the glioma cells to imidazole ketone erastin (IKE), resulting in significant increase in lipid peroxidation. Dose response cell viability assays also were performed, which again showed that CMD sensitized glioma cells to IKE. Both these effects were rescued by ferrostatin.

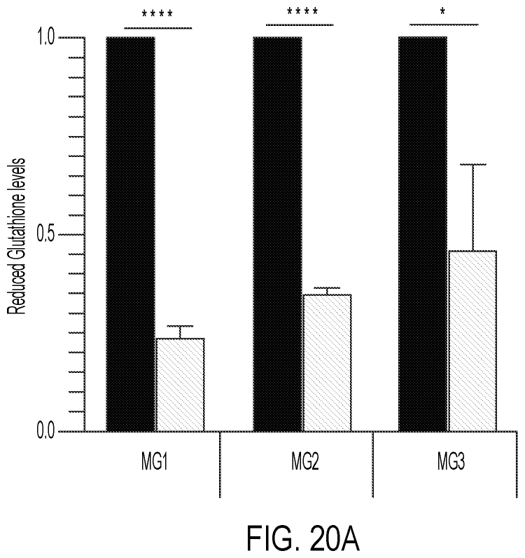

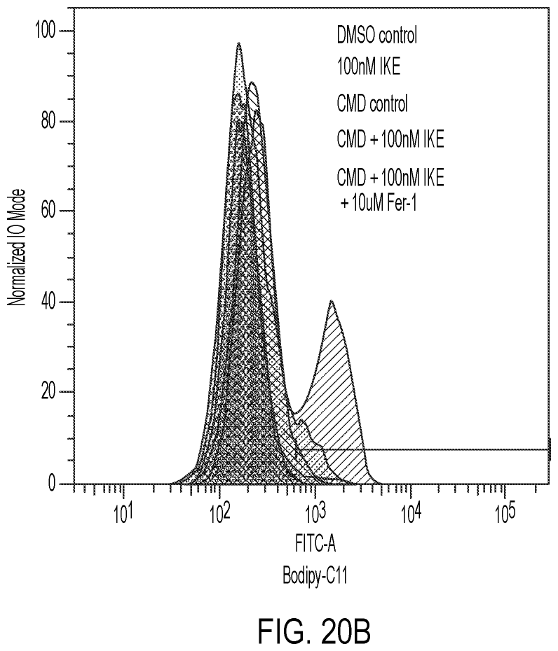

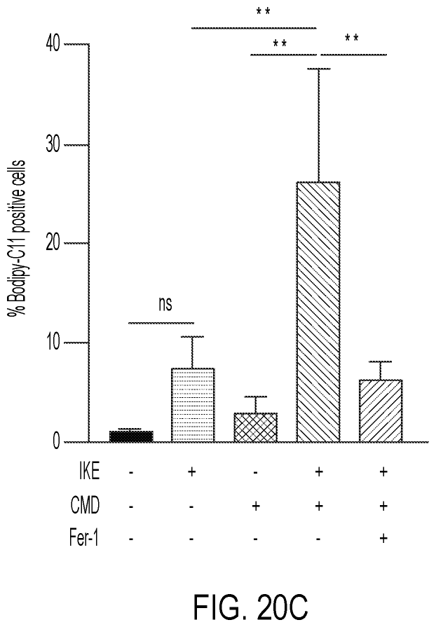

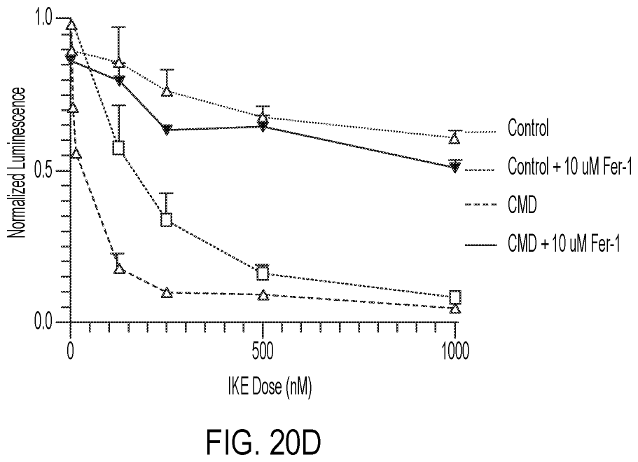

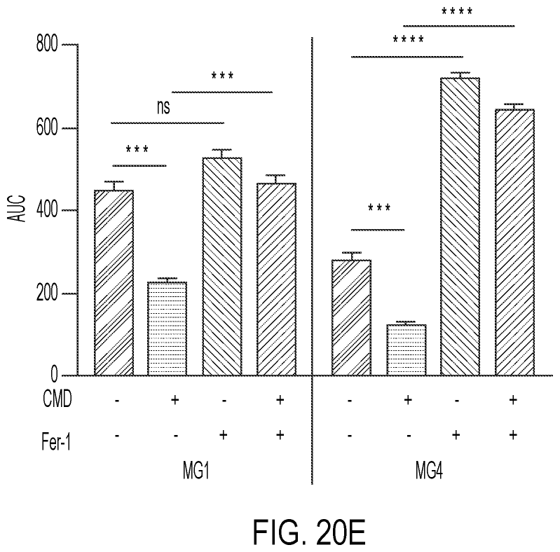

FIG. 20A shows the results of in vitro experiment demonstrating that growing cells in CMD lead to lower levels of GSH, including glutathione levels for 3 mouse glioma cell lines MG1, MG2, MG3, for control conditions (black bars) or 24 hours of CMD (gray bars). FIG. 20B shows representative Bodipy-C11 flow data from MG1 cells: DMSO control (red), 100 nM IKE (green), CMD control (orange), CMD+100 nM IKE (blue) and CMD+100 nM IKE+10 uM Ferrostatin-1 (cyan). FIG. 20C presents the quantitation of 3 independent flow cytometry experiments. FIG. 20D presents close response curves showing the effects of IKE (48 hours) on mouse glioma cell viability in different media conditions: control (blue), control+10 uM Ferrostatin-1(red), CMD (green), and CMD+10 uM Ferrostatin-1 (black). FIG. 20E contains AUC analysis of close response for 2 mouse glioma cell lines and shows that CMD causes a significant enhancement of IKE induced cell death, which is rescued by ferrostatin. Thus, CMD lowers GSH levels and sensitizes cells to imidazole ketone erastin (IKE)-induced lipid peroxidation and ferroptosis.

### Example 8. CMD Synergizes with Radiation to Induce Cell Death and Lipid Peroxidation

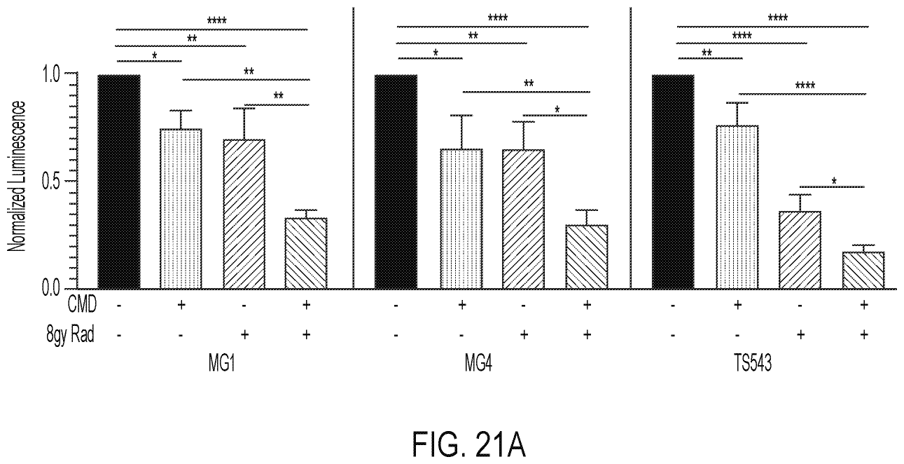

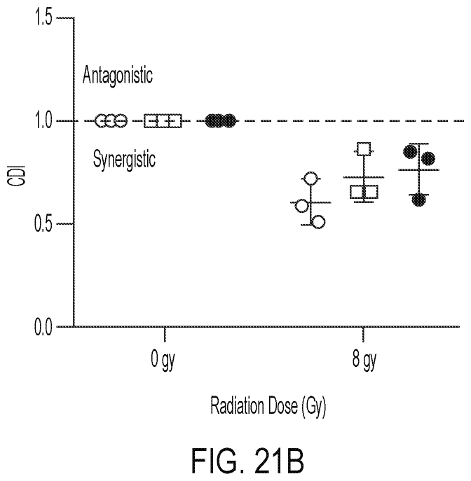

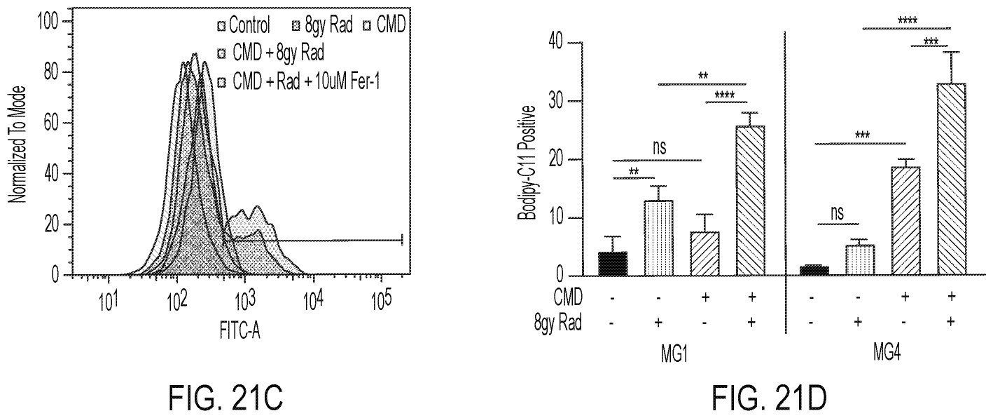

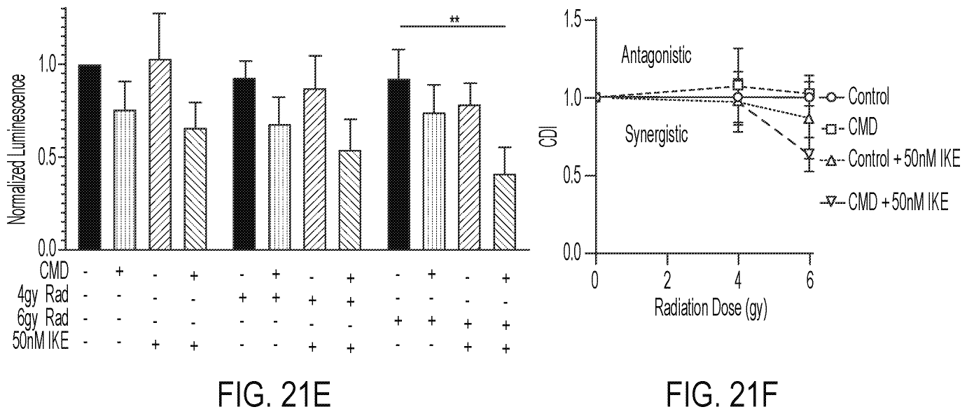

Radiation is a known ferroptosis inducer. Based on this, the CMD diet was tested to determine whether it would synergize with radiation to induce lipid peroxidation and ferroptotic cell death. Cells were treated with a combination of CMD and radiation and assayed for cell viability via bio-luminescence assays and for levels of lipid peroxidation using flow cytometry quantification of Bodipy C-11 fluorescence. FIG. 21A shows the quantitation of cell viability 120 hours after treatment with either control, CMD alone, 8 Gy irradiation alone, or CMD plus 8 Gy irradiation. Treatment of both mouse glioma cells (MG1 and MG4) and human GBM cells (TS543) is shown. FIG. 21B shows the coefficient of drug interaction (CDI) quantitation for the cell viability data (CDI=AB/AxB), with CDI<1.0 indicating synergy between CMD and radiation. FIG. 21C presents representative Bodipy-C11 flow cytometry data from MG4 cells showing increased lipid peroxidation with co-treatment of radiation plus CMD and complete rescue with Ferrostatin-1. FIG. 21D shows the quantitation of 3 independent experiments of Bodipy-C11 lipid peroxidation in MG1, MG4 cells.

FIG. 21E shows quantitation of cell viability following 72 hours of treatment across 2 radiation closes and 4 conditions: control, CMD, control+50 nM IKE, CMD+50 nM IKE. FIG. 21F presents the coefficient of drug interaction quantification for the cell viability data presented in FIG. 21E. Data in FIG. 21 are plotted as the mean t SEM; n=3 independent experiments for A, D. *p<0.05, **p<0.01, ***p<0.001, ****p<00.0001. Statistics assessed using a one-way ANOVA.

### Example 9. CMD Treatment in Combination with Radiation

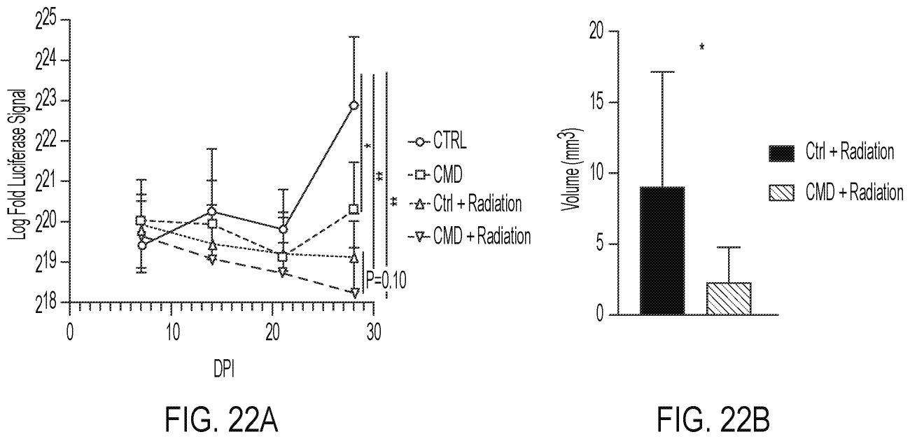

FIG. 22 shows serial luciferase imaging performed on mice bearing orthotopically injected low grade glioma cells (MG1 cells). Mice underwent orthotopic injections with MG1 mouse glioma cells using a Jackson stereotactic frame for reproducible targeting of glioma cells to the subcortical white matter. Following injection with 50,000 cells, mice were randomized into 4 treatment groups. The mice were transitioned to a CMD or Control diet on day post injection 7. Mice were further separated into sham or radiation treatment arms with treatment occurring at day post injection 21. Luciferase imaging was performed on all mice weekly all 4 groups.

FIG. 22 provides data showing that CMD treatment in combination with radiation results in decreased rate of tumor growth in vivo as determined by luciferase imaging measuring tumor volume. This finding demonstrates that combined treatment with CMD and radiation leads to a measurable in-vivo effect in an orthotopic glioma model.

### Example 10. CMD Treatment and Radiation Enhance Tumor Killing

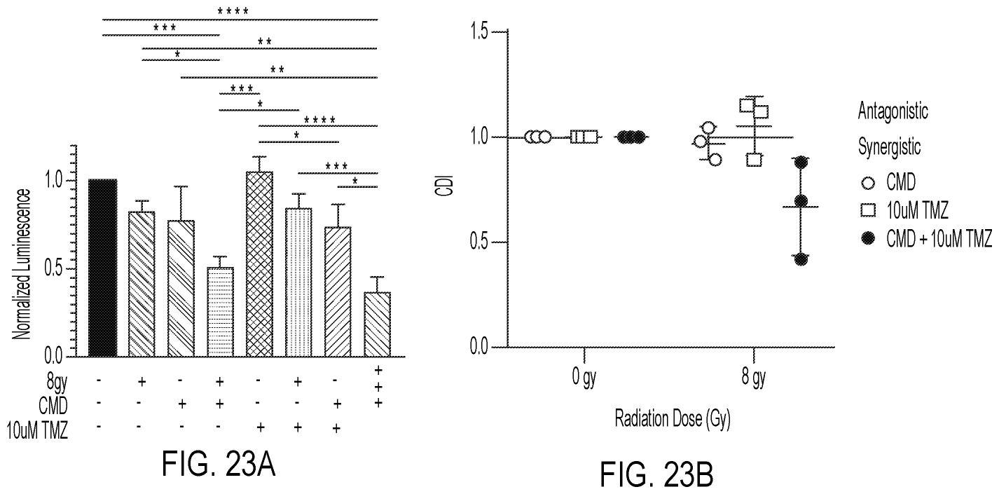

Similar to FIG. 21, FIG. 23A shows cell viability of mouse glioma cells (MG4 cells) treated with combinations of cysteine methionine deprivation, radiation or temozolomide. Cell viability was quantitated after 120 hours of treatment with the various combinations of treatments. FIG. 21B shows the coefficient of drug interaction (CDI) quantitation for the cell viability data (CDI=AB/AxB), with CDI<1.0 indicating synergy between CMD and radiation.

FIG. 23A provides data showing that CMD and radiation combined with temozolomide enhance tumor killing in vitro. FIG. 23B shows CMD plus temozolomide synergizes with radiation to induce more cell death than expected.

### Example 11. CMD and Radiation Improve Survival in Vivo

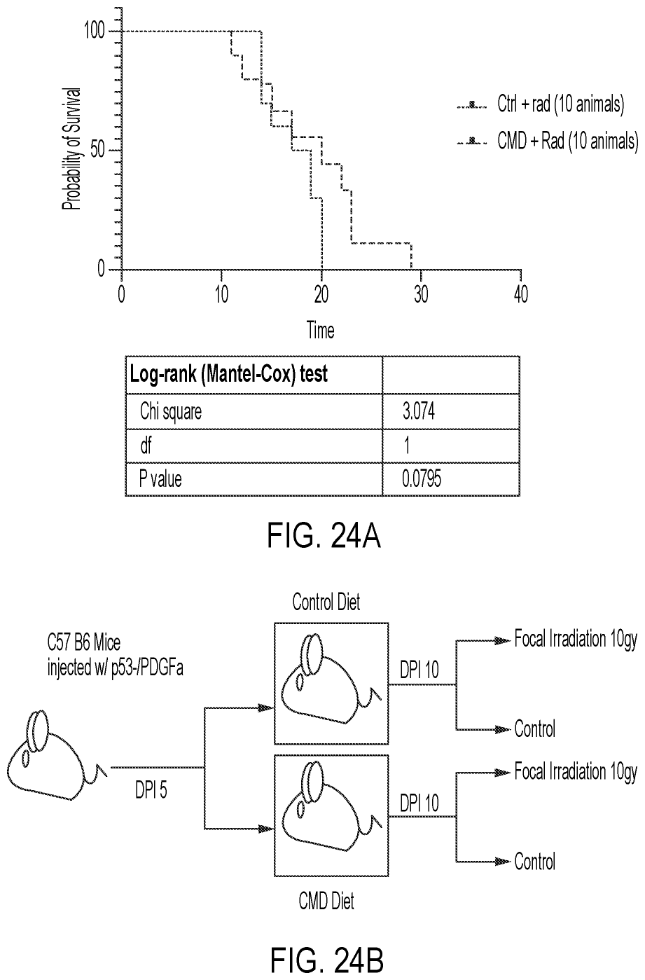

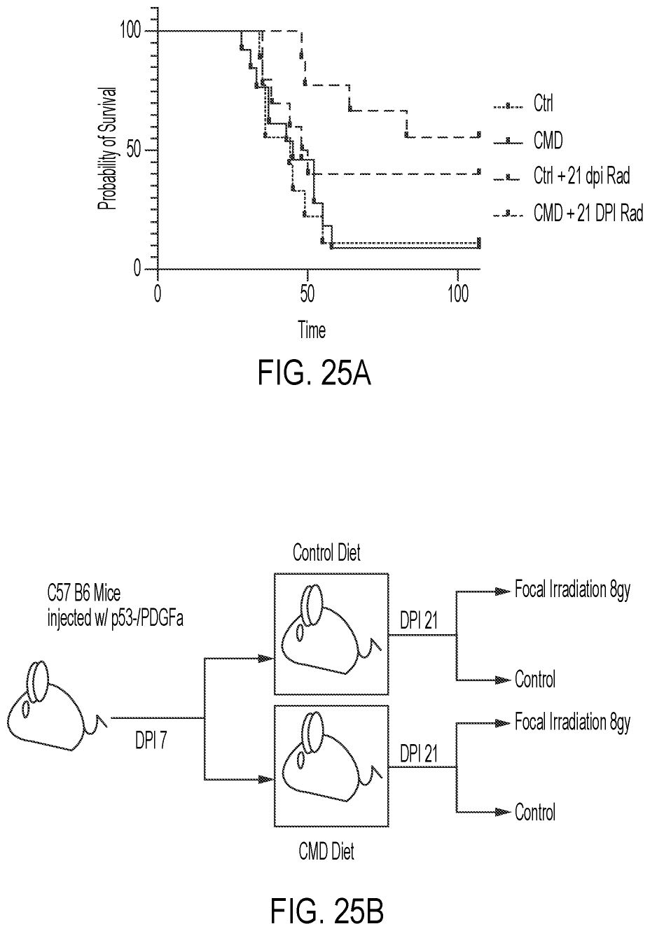

FIG. 24 and FIG. 25 outline in vivo experiments examining the efficacy of the CMD diet plus stereotactic radiation treatment in a high grade (FIG. 24, MG4 cells) and a low-grade (FIG. 25, MG1 cells) model. Mice underwent orthotopic injections of glioma cells followed by transition to a CMD diet either 5 days (FIG. 24, MG4 cells) or 7 days (FIG. 25, MG1 cells) after cell implantation. Then mice were radiated depending on tumor type. Survival data from these experiments are outlined.

CMD and radiation improve survival in vivo in both the high grade mouse glioma model (see FIG. 24) and in a low grade glioma model (see FIG. 25). These findings highlight the potential for CMD to synergize with standard of care radiation in various glioma subtypes.

### Example 12. Human Diffuse Astrocytoma Slice Cultures

To further understand the translational relevance of these findings to human glioma samples, an acute organotypic slice culture model was used. Human surgical specimens were collected from Columbia University Medical Center operating theaters in accordance with Institutional Review Board protocols. Surgical specimens were deidentified, placed in a sterile 50 mL conical tube containing the ice-cold sucrose solution. The tissue sections were cut into 300-500 μM sections using a McIlwain™ Tissue Chopper. The tissue was placed on a Millicell™ cell culture insert in treatment media. This media comprised of DMEM+ Hams-F12 without cysteine or methionine (MyBioSource, MBS652871) or DMEM+ Hams-F12 with cysteine and methione to create the to varied conditions that would undergo radiation or IKE treatment.

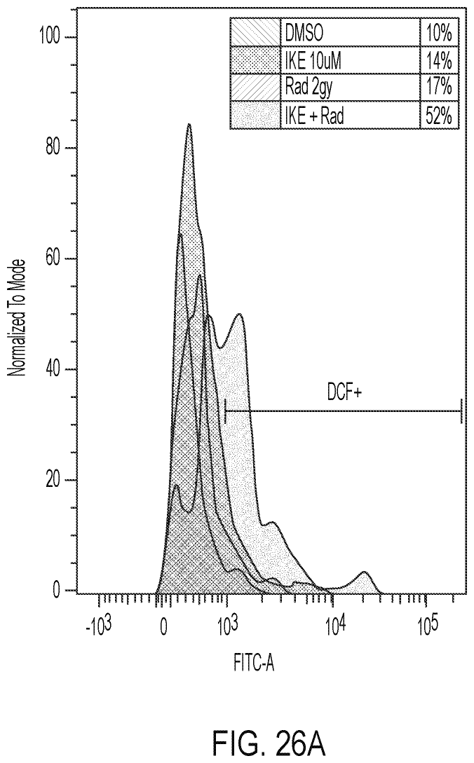

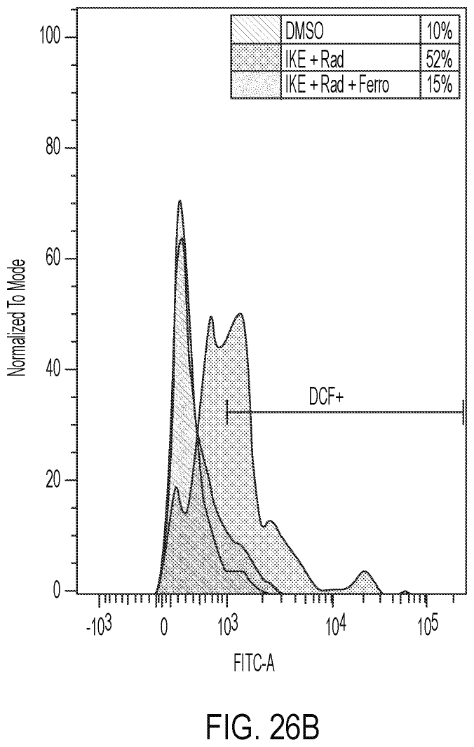

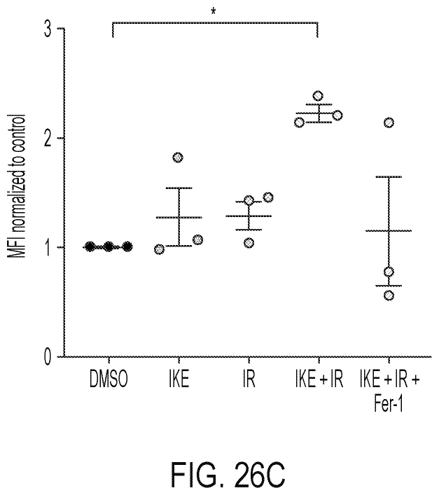

The representative histograms of FIG. 26A and FIG. 26B show a human diffuse astrocytoma slice culture sample treated with DMSO, 10 μM IKE, or 10 μM IKE+10 μM ferrostatin-1, co-treated with 0 or 2 Gy radiation for 24 hours, dissociated, stained with H2DCFDA, and measured by flow cytometry showing synergy of radiation with IKE treatment. Horizontal bars indicate H2DCFDA-positive cell populations. FIG. 26C shows the H2DCFDA staining of three human glioma slice culture samples treated with same conditions. *p<0.05. Table 5, below shows the characteristics of gliomas from which the slice cultures were derived.

TABLE 5
Characteristics of Human Gliomas.

| | | | |
|---|---|---|---|
| Tumor | | | |
| Bank ID | Age | Sex | Diagnosis |

Positive response to radiation

| | | | |
|---|---|---|---|
| 6163 | 23 | M | Diffuse astrocytoma, grade II |
| 6177 | 52 | M | Anaplastic astrocytoma, grade III |
| 6181 | 32 | F | Anaplastic oligodendroglioma, grade III |

Negative response to radiation

| | | | |
|---|---|---|---|
| 6186 | 66 | M | Glioblastoma, grade IV |
| 6193 | 67 | M | Glioblastoma, grade IV |

### Example 13. Radiation Plus IKE Treatment Increases Cancer Cell Death

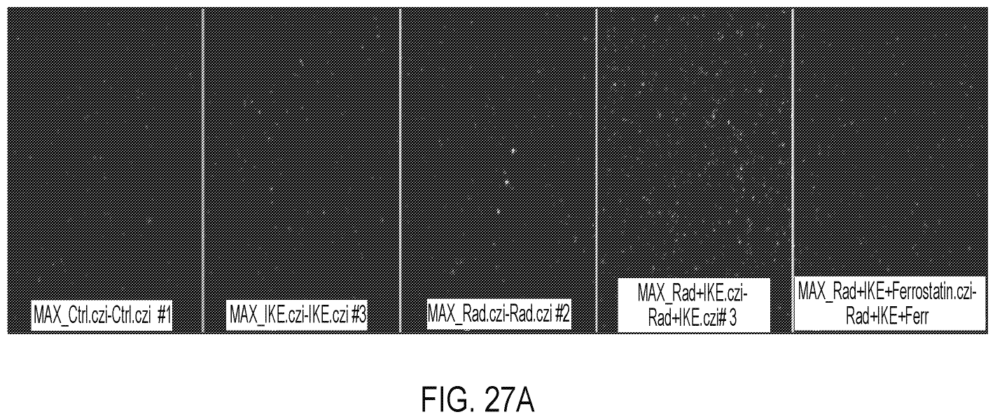

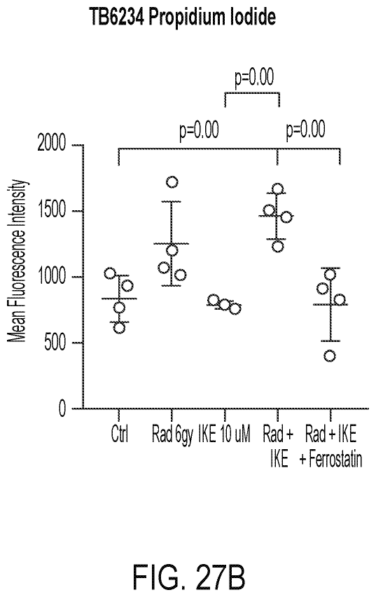

Ex vivo organotypic slices of the gliomas in Table 5, above, were plated into control, IKE or IKE+ferrostatin conditions. At t=24 hours, they were treated with either 0 gy or 6 gy radiation. At t=48 hours, slices were stained with propidium iodide and 4 z-stacks were imaged on a Zeiss™ confocal microscope. Maximum projects from 4 random areas of each slice were quantitated for mean fluorescence intensity. See FIG. 27.

### Example 14. Tumor Viability with Altered Cysteine and Methionine Concentrations

To help determine the effects of varied concentrations of cysteine and methionine, the levels of cysteine and methionine in the basal media were altered using volume dilutions. With our control media representing 100% cysteine and 100% methionine, we made various culture media depriving just one or both amino acids. Cells were treated for 24 hours in the culture media plus a treatment drug including a known ferroptosis inducer, RSL-3, with a glutathione analog/possible ferroptosis inhibitor N-acetylcysteine, or the combination of both. The effect was quantified using bioluminescence assays to assess cell viability.

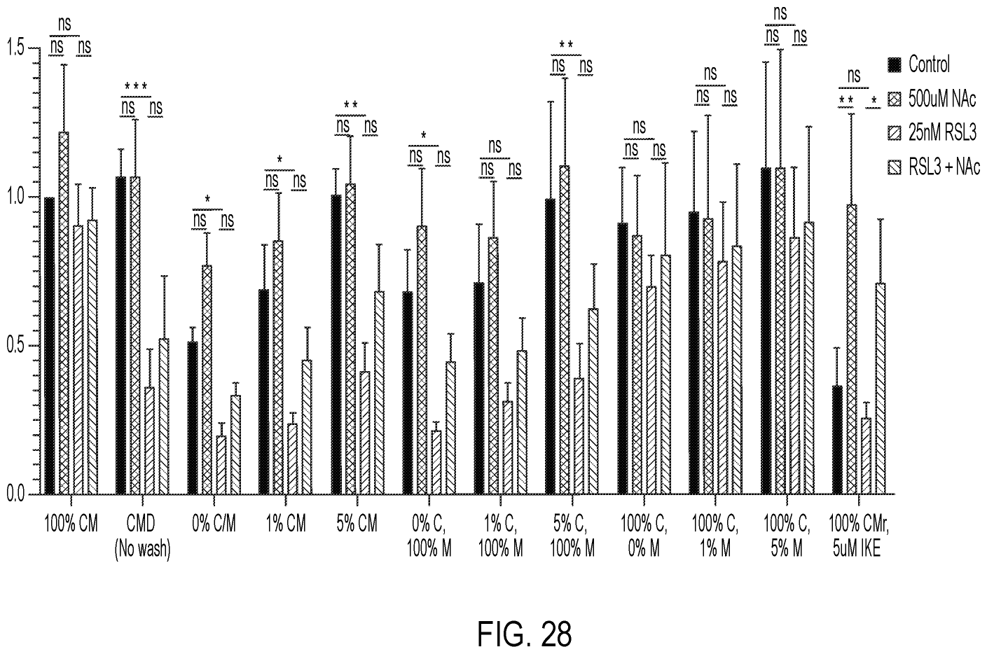

FIG. 28 shows that altering cysteine and methionine concentrations alters tumor viability and susceptibility to ferroptosis in vitro. These findings showed that cysteine deprivation is more responsible for the acute induction of ferroptosis and highlights the possible role of methionine as a sulfure store for conversion to cysteine.

### Example 15. Synergism with Radiation Treatment and Cancer Chemotherapy

The data presented here indicates that the CMD dietary formulation and methods according to the invention synergize with radiation treatment and specific chemotherapy type drugs in mouse and human glioma cells. Mouse cells were plated at a density of 4,000 cells per well and human cells plated at a density of 2,000 cells per well in 96 well plates. Twenty-four hours after plating, cells were switched into treatment media (control or CMD) and treated with radiation using a Gammacell 40 Caesium 137 irradiator (Theratronics™) and incubated for either 120 hours. All cell viability assays as described above were quantified using Cell-Titer Glo (Promega™) ATP based bioluminescence. To determine cell viability, a 50% Cell Titer Glo and 50% cell culture medium were added to each well and incubated at room temperature for 10 minutes. Luminescence was assessed on a Promega™ GloMax Microplate Reader.

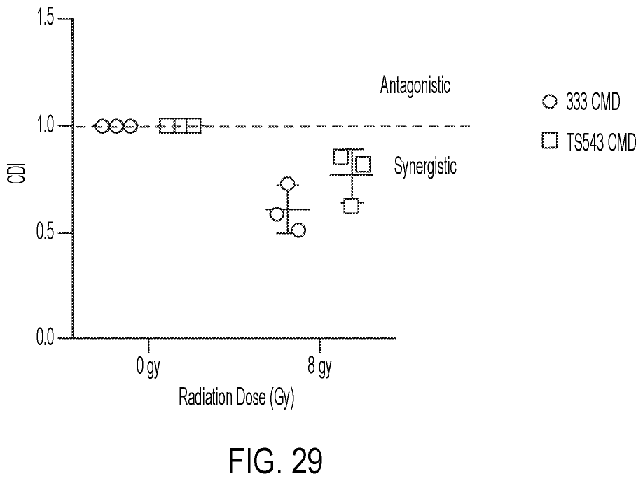

FIG. 29 shows the calculation of synergy when mouse cells (333) and human cells (TS543) are treated with both cysteine/methionine deprivation (CMD) and radiation. Values less than 1 signify synergy or greater than additive effects. Thus, this diet synergizes with radiation treatment in mouse and human glioma cells. See FIG. 29.

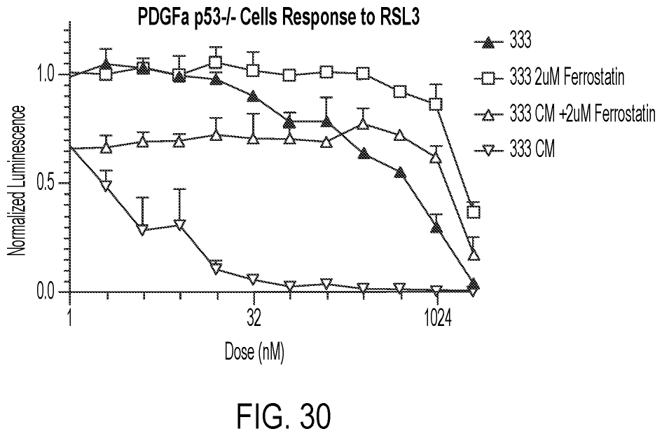

FIG. 30 shows close response curves combining CMD with a chemotherapy compound (RSL3, a ferroptosis inducer). The close response curve shows a stark difference in cell viability with dual treatment. See FIG. 30. This mechanism likely occurs through a depletion of glutathione, an antioxidant that reduces cancer cell death due to radiation and various chemotherapeutics.

### Example 16. Reduction of Glioma Growth Rate and Survival Increase In Vivo

To understand the effects of the CMD diet in vivo, a low-grade orthotopic glioma model (MG3) was used for evaluation of survival. Mice underwent orthotopic injections with MG3 mouse glioma cells using a Jackson™ stereotactic frame for reproducible targeting of glioma cells to the subcortical white matter. After injection, mice were randomized into groups and received a control diet or a CMD diet 7 days following cell implantation. These diets were formulated to have controlled levels of all macro and micronutrients based on weight/weight values except for cysteine and methionine; while the control diet has 0.43% methionine and 0.33% cystine, the CMD diet had 0.15% methionine and 0.0% cysteine.

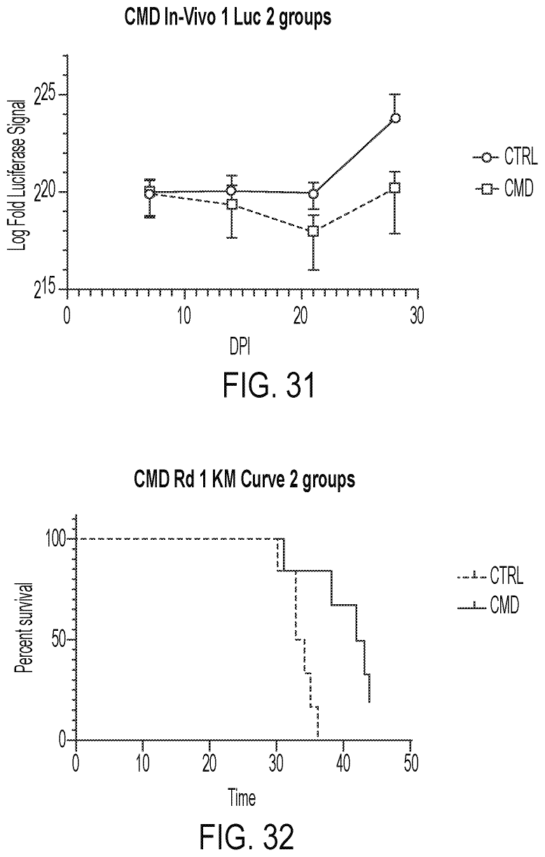

Thus, the in vivo mouse study where a cysteine depleted/methionine restricted diet is well tolerated, decreases the rate of glioma growth, and can increase survival of orthotopic glioma tumor bearing mice with no observable toxicity. See FIG. 31, which shows that glioma growth is slowed in vivo when mice are placed on a cysteine deprived/methionine restricted diet, and FIG. 32, which shows that a cysteine deprived/methionine restricted diet improved survival in a mouse model of diffusely infiltrating glioma. Taken together these findings show that methods according to embodiments of the invention are promising for use in cancer patients.

### REFERENCES

All references listed below and throughout the specification are hereby incorporated by reference in their entirety.
1. Gilbert, M. R. et al. A randomized trial of bevacizumab for newly diagnosed glioblastoma. N. Engl. J. Med. 370, 699-708 (2014).
2. Chinot, O. L. et al. Bevacizumab plus radiotherapy-temozolomide for newly diagnosed glioblastoma. N. Engl. J. Med. 370, 709-722 (2014).
3. Robert. S. M. et al. SLC7A 11 Expression Is Associated With Seizures and Predicts Poor Survival in Patients With Malignant Glioma. Sci. Transl. Med. 7, (201S).
4. Liang. J. et al. Mitochondrial PKM2 regulates oxidative stress-induced apoptosis by stabilizing Bcl2. Cell Res. 27, 329-3S1 (2017).
5. Zheng. L. et al. JNK Activation Contributes to Oxidative Stress-Induced Parthanatos in Glioma Cells via Increase of Intracellular ROS Production. Mol. Neurobiol. 54, 3492-3SOS (2017).
6. Hadian, K. & Stockwell. B. R. SnapShot: Ferroptosis. Cell 181, 1188-1188.e1 (2020).
7. Yang. W. S. & Stockwell. B. R. Ferroptosis: Death by Lipid Peroxidation. Trends Cell Biol. 26, 16S-176 (2016).
8. Yang, W. S. et al. Regulation of ferroptotic cancer cell death by GPX4. Cell 156, 317-331 (2014).
9. Dixon. S. J. et al. Ferroptosis: an iron-dependent form of nonapoptotic cell death. Cell 149, 1060-1072 (2012).
10. Yu, X. & Long. Y. C. Crosstalk between cystine and glutathione is critical for the regulation of amino acid signaling pathways and ferroptosis. Sci. Rep. 6, 30033 (2016).
11. Hayano. M., Yang. W. S., Corn. C. K., Pagano. N. C. & Stockwell, B. R. Loss of cysteinyl-tRNA synthetase (CARS) induces the transsulfuration pathway and inhibits ferroptosis induced by cystine deprivation. Cell Death Differ. 23, 270-278 (2016).
12. Shimada. K., Hayano, M., Pagano, N. C. & Stockwell. B. R. Cell-Line Selectivity Improves the Predictive Power of Pharmacogenomic Analyses and Helps Identify NADPH as Biomarker for Ferroptosis Sensitivity. Cell Chem Biol 23, 22S-23S (2016).
13. Ye, L. F. et al. Radiation-Induced Lipid Peroxidation Triggers Ferroptosis and Synergizes with Ferroptosis Inducers. ACS Chem. Biol. 15, 469-484 (2020).
14. Badgley, M. A. et al. Cysteine depletion induces pancreatic tumor ferroptosis in mice. Science 368, 8S-89 (2020).
15. Gao, X. et al. Ibuprofen induces ferroptosis of glioblastoma cells via downregulation of nuclear factor erythroid 2-related factor 2 signaling pathway. Anticancer Drugs 31, 27-34 (2020).
16. Zhao, W. et al. Deconvolution of cell type-specific drug responses in human tumor tissue with single-cell RNA-seq. Genome Med. 13.82 (2021).
17. Chen, M.-S. et al. CHAC1 degradation of glutathione enhances cystine-starvation-induced necroptosis and ferroptosis in human triple negative breast cancer cells via the GCN2-eIF2a-ATF4 pathway. Oncotarget 8, 114S88-114602 (2017).
18. Wang, N., Zeng, G.-Z., Yin, J.-L. & Bian, Z.-X. Artesunate activates the ATF4-CHOP-CHAC1 pathway and affects ferroptosis in Burkitt's Lymphoma. Biochem. Biophys. Res. Commun. 519. S33-S39 (2019).
19. Fujii, J., Homma. T. & Kobayashi. S. Ferroptosis caused by cysteine insufficiency and oxidative insult. Free Radic. Res. 1-12 (2019).
20. van der Reest, J., Lilla, S., Zheng, L., Zanivan, S. & Gottlieb, E. Proteome-wide analysis of cysteine oxidation reveals metabolic sensitivity to redox stress. Nat. Commun. 9, 1S81 (2018).
21. Yu, H., Guo, P., Xie, X., Wang, Y. & Chen, G. Ferroptosis, a new form of cell death, and its relationships with tumourous diseases. J. Cell. Mol. Med. 21, 648-6S7 (2017).
22. Liu, D. S. et al. Inhibiting the system x/glutathione axis selectively targets cancers with mutant-pS3 accumulation. Nat. Commun. 8, 14844 (2017).
23. Stockwell. B. R. et al. Ferroptosis: A Regulated Cell Death Nexus Linking Metabolism, Redox Biology, and Disease. Cell 171, 273-28S (2017).
24. Hancock. S. E., Friedrich. M. G., Mitchell. T. W., Truscott, R. J. W. & Else. P. L. The phospholipid composition of the human entorhinal cortex remains relatively stable over 80 years of adult aging. Geroscience 39, 73-82 (2017).
25. Zhang. Y. et al. Imidazole Ketone Erastin Induces Ferroptosis and Slows Tumor Growth in a Mouse Lymphoma Model. Cell Chem Biol 26, 623-633.e9 (2019).
26. Tang. D., Chen, X., Kang, R. & Kroemer, G. Ferroptosis: molecular mechanisms and health implications. Cell Res. 31, 107-12S (2020).
27. Yang. B. et al. w-6 Polyunsaturated fatty acids (linoleic acid) activate both autophagy and antioxidation in a synergistic feedback loop via TOR-dependent and TOR-independent signaling pathways. Cell Death Dis. 11, 607 (2020).
28. Mukherjee. P. et al. Therapeutic benefit of combining calorie-restricted ketogenic diet and glutamine targeting in late-stage experimental glioblastoma. Commun Biol 2, 200 (2019).
29. Martuscello, R. T. et al. A Supplemented High-Fat Low-Carbohydrate Diet for the Treatment of Glioblastoma. Clin. Cancer Res. 22, 2482-249S (2016).
30. Jiang, Y.-S. & Wang, F.-R. Caloric restriction reduces edema and prolongs survival in a mouse glioma model. J. Neurooncol. 114, 2S-32 (2013).
31. Weissenberger. J. et al. Dietary curcumin attenuates glioma growth in a syngeneic mouse model by inhibition of the JAK1.2/STAT3 signaling pathway. Clin. Cancer Res. 16, S781-S79S (2010).
32. Zhou, W. et al. The calorically restricted ketogenic diet, an effective alternative therapy for malignant brain cancer. Nutr. Metab. 4. S (2007).
33. Marsh, J.. Mukherjee. P. & Seyfried, T. N. Akt-dependent proapoptotic effects of dietary restriction on late-stage management of a phosphatase and tensin homologue/tuberous sclerosis complex 2-deficient mouse astrocytoma. Clin. Cancer Res. 14, 77S1-7762 (2008).
34. Chen. L. et al. Erastin sensitizes glioblastoma cells to temozolomide by restraining xCT and cystathionine-γ-lyase function. Oncol. Rep. 33, 146S-1474 (201S).
35. Sonabend, A. M. et al. Murine cell line model of proneural glioma for evaluation of anti-tumor therapies. J. Neurooncol. 112, 37S-382 (2013).
36. Montgomery, M. K. et al. Glioma-Induced Alterations in Neuronal Activity and Neurovascular Coupling during Disease Progression. Cell Rep. 31, 107S00 (2020).
37. Szerlip, N. J. et al. Intratumoral heterogeneity of receptor tyrosine kinases EGFR and PDGFRA amplification in glioblastoma defines subpopulations with distinct growth factor response. Proc. Natl. Acad. Sci. U.S.A 109, 3041-3046 (2012).
38. Takeshita, I. et al. Characteristics of an established human glioma cell line, KNS-42. Neurol. Med. Chir. 27. S81-S87 (1987).
39. Wang, X., Spandidos, A., Wang, H. & Seed. B. PrimerBank: a PCR primer database for quantitative gene expression analysis, 2012 update. Nucleic Acids Res. 40. D1144-D1149 (2012).
40. Nguyen. T. T. T. et al. HDAC inhibitors elicit metabolic reprogramming by targeting super-enhancers in glioblastoma models. J. Clin. Invest. 130, 3699-3716 (2020).
41. Sonabend, A. M. et al. Convection-enhanced delivery of etoposide is effective against murine proneural glioblastoma. Neuro. Oncol. 16, 1210-1219 (2014).
42. Amino Acid Restriction Triggers Angiogenesis via GCN2/ATF4 Regulation of VEGF and H2S Production. Cell 173, 117-129.e14 (2018).
43. Chong. J. et al. MetaboAnalyst 4.0: towards more transparent and integrative metabolomics analysis. Nucleic Acids Res. 46, W486-W494 (2018).
44. Marchione, D. M. et al. HYPERsol: High-Quality Data from Archival FFPE Tissue for Clinical Proteomics. J. Poteome Res. 19, 973-983 (2020).
45. Nickerson. J. L. & Doucette, A. A. Rapid and Quantitative Protein Precipitation for Proteome Analysis by Mass Spectrometry. J. Proteome Res. 19, 2035-2042 (2020).
46. Kulak. N. A., Pichler. G., Paron, I., Nagaraj, N. & Mann. M. Minimal, encapsulated proteomic-sample processing applied to copy-number estimation in eukaryotic cells. Nat. Methods 11, 319-324 (2014).
47. Bruderer, R. et al. Extending the limits of quantitative proteome profiling with data-independent acquisition and application to acetaminophen-treated three-dimensional liver microtissues. Mol. Cell. Proteomics 14, 1400-1410 (2015).
48. Kang J S. Dietary restriction of amino acids for cancer therapy. Nutr Metab (Lond). 2020; 17.
49. Tajan M, Vousden K H. Dietary approaches to cancer therapy. Cancer Cell. 2020 Jun. 8; 37(6): pp. 767-785.
50. Hassannia B, Vandenabeele P, Berghe T V. Targeting ferroptosis to iron out cancer. Cancer Cell. 2019 Jun. 10; 35(6): pp. 830-49.

1. A method of treating cancer in a subject in need thereof, the method comprising administering a cysteine and methionine deprivation (CMD) diet to the cancer subject.

2. The method of claim 1, wherein the CMD diet comprises a CMD formulation.

3. The method of claim 2, wherein the CMD formulation comprises:
(a) about 4% to about 60% fat by weight;
(b) about 24% to about 73% carbohydrate by weight;
(c) about 10% to about 25% protein by weight;
(d) about 0% vitamin E by weight;
(e) about 0% cysteine by weight;
(f) about 0% to about 0.15% methionine by weight;
(g) about 0% selenium by weight;
(h) about 0% to about 10% saturated fatty acids by weight;
(i) about 18 mg to about 65 mg iron per daily serving; and
(j) about 0 g to about 50 g alanyl-glutamine per daily serving,
wherein the poly unsaturated fatty acid (PUFA) to monounsaturated fatty acid (MUFA) ratio is at least 2:1.

4. The method of claim 3, wherein the CMD formulation comprises about 25% fat, about 53% carbohydrate, about 15% protein, and about 40 g per daily serving alanyl-glutamine.

5. The method of claim 1, further comprising co-administering radiotherapy to the subject in need.

6. The method of claim 5, wherein the CMD diet and radiotherapy synergistically kill cancer cells.

7. The method of claim 5, wherein the CMD diet reduces the coefficient of drug interaction of a given radiation dose of the radiotherapy.

8. The method of claim 1, wherein the CMD diet promotes iron ferroptosis in cancer cells in the subject.

9. The method of claim 1, further comprising co-administering to the subject a chemotherapeutic agent.

10. The method of claim 9, wherein the chemotherapeutic agent promotes iron ferroptosis in cancer cells in the subject.

11. A dietary formulation to reduce cysteine and/or methionine in a subject in need, comprising
(a) about 4% to about 60% fat by weight;
(b) about 24% to about 73% carbohydrate by weight;
(c) about 10% to about 25% protein by weight;
(d) about 0% vitamin E by weight;
(e) about 0% cysteine by weight;
(f) about 0% to about 0.15% methionine by weight;
(g) about 0% selenium by weight;
(h) about 0% to about 10% saturated fatty acids by weight;
(i) about 18 mg to about 65 mg iron per daily serving; and
(j) about 0 g to about 50 g alanyl-glutamine per daily serving,
wherein the poly unsaturated fatty acid (PUFA) to monounsaturated fatty acid (MUFA) ratio is at least 2:1.

12. The method of claim 11, wherein the dietary formulation comprises about 25% fat, about 53% carbohydrate, about 15% protein, and about 40 g per daily serving alanyl-glutamine.

13. The dietary formulation of claim 10, which is in the form of a food product for oral consumption.

14. An article of manufacture comprising a container and the dietary formulation of claim 11 disposed therein.

15. An article of manufacture comprising a container and the dietary formulation of claim 12 disposed therein.


SEQUENCE LISTING (16 sequences)

SEQ ID NO: 1 - DNA, 21 bp, Artificial Sequence
  Synthetic
catgtacgttgctatccaggc

SEQ ID NO: 2 - DNA, 20 bp, Artificial Sequence
  Synthetic
ctccttaatgtcacgacgat

SEQ ID NO: 3 - DNA, 20 bp, Artificial Sequence
  Synthetic
tctccaaaggaggttactgc

SEQ ID NO: 4 - DNA, 22 bp, Artificial Sequence
  Synthetic
agactcccctcagtaaagtgac

SEQ ID NO: 5 - DNA, 21 bp, Artificial Sequence
  Synthetic
atgaccgaaatgagcttcctg

SEQ ID NO: 6 - DNA, 19 bp, Artificial Sequence
  Synthetic
gctggagaacccatgaggt

SEQ ID NO: 7 - DNA, 20 bp, Artificial Sequence
  Synthetic
cgaggcccagagcaagagag

SEQ ID NO: 8 - DNA, 21 bp, Artificial Sequence
  Synthetic
ctcgtagatgggcacagtgtg

SEQ ID NO: 9 - DNA, 20 bp, Artificial Sequence
  Synthetic
cctgaacagcgaagtgttgg

SEQ ID NO: 10 - DNA, 22 bp, Artificial Sequence
  Synthetic
tggagaacccatgaggtttcaa

SEQ ID NO: 11 - DNA, 19 bp, Artificial Sequence
  Synthetic
ggcaccgtcatcggatcag

SEQ ID NO: 12 - DNA, 21 bp, Artificial Sequence
  Synthetic
ctccacaggcagaccagaaaa

SEQ ID NO: 13 - DNA, 22 bp, Artificial Sequence
  Synthetic
ttcaacacactctatcactggc

SEQ ID NO: 14 - DNA, 21 bp, Artificial Sequence
  Synthetic
agaagcgtttgcggtactcat

SEQ ID NO: 15 - DNA, 20 bp, Artificial Sequence
  Synthetic
ctgtggattttcgggtacgg

SEQ ID NO: 16 - DNA, 19 bp, Artificial Sequence
  Synthetic
ccctatggaaggtgtctcc